SEQUENCE LISTING (6 sequences)

SEQ ID NO: 1 - DNA, 862 bp, Pteris vittata
aatcattttggatatgtcaaatattaaattttatgacatgacgtatgcgacatagagttaatgcaactgctcccaatatatacattactaaaagtagacagtgtgatagtgctgccccatccctctcgttatatcccgttctgaacacaggatttgtgatgaatatacttggattatccaaggccgatgccaacattacctatgcctgactgccagccaagtaacacagcttatgtgtggcagtgtcagagagtggtcgtagtgagggtattgtatagagtagtactagatataggaagtatgtattttcctatggggccaaggtttctaaatctgtgtgtggtattaactatacactatggataacgaagacagttaattttcagaagatattaaacaacgcgaaaattcagtagtagcgaatgtcgattgttagtgcatgcacctcagtagggatcagaccctacttgtgtggttgtgttggatgccatttgcgagtttgcaatgtcagcacttcagttgctaccaccgattattcgatatgtttgtcaaagctgttgtatatcgatgcgtgtgtagtctttttcccgcccaggatgataaagatgtgggttgatccacgcgcgcacgtgcgtgtgtgtgtgagataggtagattgaactgcacggggggcaatgtccttgcagtgattgattagtatgcgtgtgagggttaattgtgggtgtgtgagtgtgaatatactagacaaaggtgccacgtatttctgtaatacgtgttgtgaaagatgttgccagagcgtagcaggagctggaagttgtcacaatcagaagagagcagcatctacgtgcgtgcatgtggggag

SEQ ID NO: 2 - DNA, 1409 bp, Pteris vittata
atggatcaggaatggtgaaggtgagatgcttcactctctccatggtttttgtccttgcaaagtgtcaatgcatattgatttgatggttctatttctactttcttttgccctggaggtcaggctggatttgctggtgatgatgccccaagggctgttttcccaagtattgtgggtcgtccaagacacactgatgtaatggtgggaatgggacaaaaggatgcgtatgttggtgatgaggcacagtccaagcgtggtattcttacattgaagtatcctattgagcatggtattgtcactaattgggatgatatggagaagatttggcatcatactttctacaatgagcttcgtgttgcacctgaagagcatcccgttttgctcacggaagcccctctgaatcccaaggcaaatcgtgagaagatgacccagataatgtttgacacgttcaatgctccagcaatgtatgttgctatccaggctgtgctctccctatatgcgagtggaagaaccacaggtatgaatgaaatattgactgtgataactgattttgaattttgtatttgcctgcagagtaagttaactgagtttttgccccttctgtaggtatcgtgcttgattccggtgatggggtcacgcacactgtacccatctacgagggttatgcattgccacatgccatcctccgtcttgatcttgctggcagggacctgacagatgcccttatgaaaatcctgactgagagagggtattcattcacaacaacagctgaaagagaaattgttcgggatatcaaagagaagctagcatatgttgccctggattttgagcaagaactcgagacttcaaagagcagctcatctttggagaagaattatgagcttcctgatgggcaggtcatcaccatcggtgcagagcgattcagatgtccagaggttctctttcagccagctctcattgggatggaagctgcaggtatccatgagactacatacaactctatcatgaagtgtgatgtggatattcgaaaggacctgtatggcaatgttgtgcttagtggaggctccaccatgtttccgggtattgctgaccgcatgagcaaagagattactgcacttgctcctagcagcatgaagataaaggttgtggcaccaccagagaggaagtacagtgtttggattggagggtccattttggcatctttaagcacattccaacaggttctctactcagcttttgtagtcacatttattttgttatgtagtttgtggagtaacttttagtactttatgtcatgtttctgtttaaagggaaacctaattgctatttttctttgtgcttttttacagatgtggattgcaaagtctgagtatgatgagtcaggcccatccatcgtccatcgcaaatgcttc

SEQ ID NO: 3 - DNA, 17 bp, Artificial
  synthetic
atggcngayggngarga

SEQ ID NO: 4 - DNA, 21 bp, Artificial
  synthetic
gaagcayttgcgrtgsacrat

SEQ ID NO: 5 - DNA, 30 bp, Artificial
  synthetic
caattgtctatgtcaatggtgagctggtcg

SEQ ID NO: 6 - DNA, 30 bp, Artificial
  synthetic
tgaacatcactggatcaatgtcgtgaaagc

(19) United States
(12) Patent Application Publication
(10) Pub. No.: US 2012/0084884 A1
(43) Pub. Date: Apr. 5, 2012

(54) STABLY TRANSFORMED FERNS AND RELATED METHODS

(75) Inventors: Neal Stewart, Knoxville, TN (US); Muthukumar Balasubramaniam, Knoxville, TN (US); Blake Joyce, Knoxville, TN (US)

(73) Assignee: University of Tennessee Research Foundation, Knoxville, TN (US)

(21) Appl. No.: 13/253,659

(22) Filed: Oct. 5, 2011

Related U.S. Application Data
(60) Provisional application No. 61/390,097, filed on Oct. 5, 2010.

(51) Int. Cl. A01H 9/00; C12N 15/82

(52) U.S. Cl. 800/293; 800/295; 800/278; 800/294; 435/320.1

(57) ABSTRACT

Methods that provide for stable transformation of fern spores and/or protonemata by Agrobacterium- and/or by particle-mediated transformation are disclosed. Also provided are stably transformed, non-chimeric ferns.

### CROSS-REFERENCE TO RELATED APPLICATIONS

This application claims the benefit of U.S. Provisional Application No. 61/390,097, filed Oct. 5, 2010 and incorporated herein by reference in its entirety.

### BACKGROUND

Ferns span roughly 250 genera and are the most ancient extant vascular land plants. They are a great resource for studying network of genes involved in stress tolerance, arsenic hyper-accumulation (Pteris vittata—Dhankher et al., 2006), insecticidal, allelopathic (Pteridium aquilinum—Marrs et al., 2006) and antimicrobial activities (Acrosticum aureum—Lai et al., 2009). Because of the lack of transformation technique to get stable transformants, ferns' gene functions are being studied either in angiosperms such as A. thaliana (Indriolo et al., 2009) or studied transiently in ferns' gametophyte stage in their life cycle (Ma et al., 2001). This is not ideal because dicots and monocots may not be physiologically suitable to study all fern genes; likewise, fern gametophytes are only part of their life cycle and moreover, they are extremely small and are not easy to study.

Transient expression of a recombinant beta-glucuronidase reporter gene in the model fern Ceratopteris richardii gametophytes was shown by Rutherford et al (2004). No viable transgenic sporophytes were recovered by Rutherford et al. Recovery of viable transgenic sporophytes by Rutherford et al. may have been precluded in part by use of transformation vectors that expressed a chloroplast bleaching RNAi that was toxic. Indriolo et al. (Plant Cell DOI: 10.1105/tpc.109.069773) also report transient expression of a recombinant gene in Ceratopteris richardii gametophytes.

Transient transformation Adiantum spp. of prothalli cells was reported by Kawai et al. 2003. Nature 421: 287-290. This report also cited a need for stable transformation systems to continue basic molecular research in Adiantum spp.

Methods for genetic transformation filamentous fungal spores by Agrobacterium tumefaciens that use an Agrobacterium growth and Agrobacterium vir gene induction medium were disclosed by Utermark et al. Nature Protocols DOI: 10.1038/nprot.2008.83, 2008. However, filamentous fungi and ferns are organisms of entirely different kingdoms. Fungal spores and fern spores also do not enter the same life cycle stage.

Mohamed et al. (2006) and Nugent et al. (2006) respectively published related meeting and poster abstracts pertaining to transient and stable transformation of the model fern Ceratopteris richardii. Nugent et al. (2006) disclosed transient expression of reporter genes in young prothalli of Ceratopteris richardii that were microinjected or bombarded with 35S or Actin promoters driving expression of a GFP reporter gene, with the “35S promoter driving more vital and efficient expression” than the Actin promoter. Nugent et al. further indicated that stable transformation was being attempted using the 35S promoter with an Ac/Ds transposon system bombarded into newly germinating spores and that initial results of stable transformation would be reported.

### SUMMARY OF INVENTION

This invention provides for stably transformed ferns (Pteridophytes), methods for obtaining stably transformed ferns (Pteridophytes), and promoters useful of obtaining or using stably transformed ferns (Pteridophytes) for a variety of applications. Provided herein are transgenic Pteridophytes, wherein said transgenic Pteridophyte is a gametophyte or a sporophyte that is stably transformed with a recombinant DNA construct. In certain embodiments, the transgenic Pteridophyte is a fern selected from the group consisting of Polypodiopsid, Psilotopsid, Equisetopsid, and Marattiopsid ferns. In certain embodiments, the transgenic Pteridophyte is selected from the group consisting of a Polystichum spp., Asplenium spp., Onoclea spp., Pteris spp., and Adiantum spp. In certain embodiments, the transgenic Pteridophyte is selected from the group consisting of Polystichum acrostichoides, Asplenium platyneuron, Asplenium nidus, Onoclea sensibilus, Pteris vittata, Pteris cretica, Pteris ensiformis, and Adiantum raddianum. In certain embodiments, the transgenic Pteridophyte is Pteris vittata. In other embodiments, the transgenic Pteridophyte is not Ceratopteris richardii. In certain embodiments, any of the aforementioned transgenic Pteridophytes can comprise a recombinant DNA construct that comprises one or more Agrobacterium T-DNA border sequences. In certain embodiments, any of the aforementioned transgenic Pteridophytes can comprise a recombinant DNA construct that comprises a Pteris vittata actin promoter that is operably linked to a sequence that encodes an RNA and/or a protein. In certain embodiments, any of the aforementioned transgenic Pteridophytes can comprise a recombinant DNA construct that comprises a gene that confers resistance to an antibiotic or a herbicide. In certain embodiments where the transgenic Pteridophyte comprises a gene that confers resistance to a herbicide or an antibiotic, the herbicide can be selected from the group consisting of bromoxynil, dicamba, glufosinate, glyphosate, and sulfonylurea herbicides and the antibiotic can be selected from the group consisting of bleomycin, gentamycin, hygromycin, and kanamycin antibiotics. In certain embodiments, any of the aforementioned transgenic Pteridophytes can comprise a recombinant DNA construct that comprises a sensor gene, a gene that provides for removal of an environmental contaminant, a gene that provides for detoxification of an environmental contaminant, a gene that provides for a counter-selection, a gene that provides for inhibition of senescence, or a combinations of these genes. In certain embodiments, any of the aforementioned transgenic Pteridophytes can be a transgenic Pteridoid or a Ceratopteridoid ferns. In certain embodiments, any of the aforementioned transgenic Pteridoid ferns can be Pteris vittata. In certain embodiments, any of the aforementioned transgenic Ceratopteridoid ferns can be Ceratopteris thalictroides.

Also provided are methods for obtaining stably transformed ferns. In certain embodiments, methods of obtaining a transgenic Pteridophyte sporophyte that is stably transformed with a recombinant DNA construct comprising the steps of: a) introducing a recombinant DNA construct into a spore or a protonemata of a Pteridophyte; b) isolating a gametophyte obtained from said spore or protonemata that comprises said recombinant DNA construct; and, c) isolating a sporophyte obtained from said gametophyte, wherein said sporophyte is stably transformed with said recombinant DNA construct are provided. In certain embodiments of the methods, the recombinant DNA construct is introduced into said spore by Agrobacterium-mediated transformation or by particle bombardment. In certain embodiments, the recombinant DNA construct is introduced into said protonemata by particle bombardment. In certain embodiments, the recombinant DNA construct can comprise a sequence encoding a selectable marker gene that confers resistance to an agent, a sequence encoding a scoreable marker gene, or both of these sequences. In certain embodiments, the selectable marker gene that confer resistance to an agent that is an antibiotic or a herbicide. In certain embodiments, the scoreable marker gene is an enzyme, a gene that provides for a change in pigmentation, or a fluorescent protein. In certain embodiments where the recombinant DNA construct comprises a sequence encoding a selectable marker gene, the isolation in step (b) and/or (c) comprises exposure of said gametophyte and/or said sporophyte to an agent that is inhibitory to a gametophyte or a sporophyte that lacks said selectable marker gene or ii) said spore or protonemata is exposed to said agent after introduction of said recombinant DNA construct. In certain embodiments where the recombinant DNA construct comprises a sequence encoding a scoreable marker gene, the isolation in step (b), step (c), or steps (b) and (c) comprises recovery of a gametophyte and/or a sporophyte that expresses said scoreable marker gene. In certain embodiments of any of the aforementioned methods, the spores, the protonemata, or both the spores and the protonemata are cultured on a surface that is permeable and transferable during, following, or both during and following recombinant DNA construct introduction. In certain embodiments that use a permeable and transferable surface, the surface can be selected from the group consisting of a polyvinylidene fluoride (PDVF) membrane, a nitrocellulose membrane, a cellulose acetate membrane, and a polytetrafluoroethylene (PTFE) membrane. In certain embodiments of any of the aforementioned methods, the Pteridophyte can be a fern selected from the group consisting of Polypodiopsid, Psilotopsid, Equisetopsid, and Marattiopsid ferns. In certain embodiments of any of the aforementioned methods, the Pteridophyte can be selected from the group consisting of a Polystichum spp., Asplenium spp., Onoclea spp., Pteris spp., and Adiantum spp. In certain embodiments, the Pteridophyte is selected from the group consisting of Polystichum acrostichoides, Asplenium platyneuron, Asplenium nidus, Onoclea sensibilus, Pteris vittata, Pteris cretica, Pteris ensiformis, and Adiantum raddianum. In certain embodiments of any of the aforementioned methods, the Pteridophyte is Pteris vittata. In certain embodiments of any of the aforementioned methods, the Pteridophyte is not Ceratopteris richardii. In certain embodiments of any of the aforementioned methods, the recombinant DNA comprises a Pteris vittata actin promoter that is operably linked to a sequence that encodes an RNA, or that encodes an RNA that encodes a protein. In certain embodiments of any of the aforementioned methods, the transgenic Pteridophytes can be transgenic Pteridoid or Ceratopteridoid ferns. In certain embodiments of any of the aforementioned methods, the transgenic Pteridoid ferns can be Pteris vittata. In certain embodiments of any of the aforementioned methods, the transgenic Ceratopteridoid ferns can be Ceratopteris thalictroides.

A recombinant DNA construct that comprises a Pteris vittata actin promoter that is operably linked to a DNA sequence that encodes a heterologous RNA, or that encodes a heterologous RNA that encodes a heterologous protein is also provided. In certain embodiments, the promoter comprises residues 1 to 862, 250 to 862, 400 to 862, 500 to 862, or 600 to 862 of SEQ ID NO:1. In certain embodiments, the promoter has at least 85%, 90%, 95%, 98%, or 99% nucleotide sequence identity to across the entire length of the corresponding residues 1 to 862, 250 to 862, 400 to 862, 500 to 862, or 600 to 862 of SEQ ID NO:1. In certain embodiments of any of the aforementioned recombinant DNA constructs, wherein said DNA sequence encodes a heterologous RNA, a heterologous RNA that provides for inhibition of senescence, a RNA that provides for a counter-selection, or a heterologous protein selected from the group consisting of selectable marker protein, a scoreable marker protein, a protein that provides for removal of an environmental contaminant, a protein that provides for detoxification of an environmental contaminant, a protein that provides for a counter-selection, and a protein that provides for inhibition of senescence.

Also provided herein are methods of expressing a heterologous gene in a Pteridophyte, comprising the steps of: a) introducing any of the aforementioned recombinant DNA constructs of the invention a Pteridophyte spore, protonemata, gametophyte, or sporophyte, and b) isolating a spore, prothallus, gametophyte, or sporophyte obtained from step (a) that comprises said recombinant DNA construct. In certain embodiments of any of the aforementioned methods, the transgenic Pteridophytes can be transgenic Pteridoid or Ceratopteridoid ferns. In certain embodiments of any of the aforementioned methods, the transgenic Pteridoid ferns can be Pteris vittata. In certain embodiments of any of the aforementioned methods, the transgenic Ceratopteridoid ferns can be Ceratopteris thalictroides.

### BRIEF DESCRIPTION OF THE DRAWINGS

The description refers to the accompanying drawings in which like references refer to like parts throughout the several views in which

FIG. 1 shows stable expression four weeks after co-cultivation of GUSPlus (intron+apoplastic signal peptide) driven by P. vittata actin promoter::EHA105 using multisite gateway binary vector.

FIG. 2 shows chimeric and full biolistic bombardment transformation of P. vittata spores and gametophytes using 2×35S CaMV:gusA (no intron). Spore transformation was carried out at ten days post-plating to target spores and freshly germinated single-celled protonemata. Transformation of gametophytes was carried out at twenty two days and yields only chimeric single cell transformation.

FIG. 3 shows stable expression of GUSPlus reporter gene in transgenic sporophytes. A. (left panel) P. vittata and B. (left panel) C-fern using P. vittata actin promoter::GUSPlus in A. tumefaciens EHA105.

FIG. 4 shows agarose gel electrophoresis of genomic PCR of transgenic sporophytes. Samples 1 and 15 are 1 Kb ladder, 1 to 12 are transgenic P. vittata sporophytes and 17 is transgenic C-fern (Ceratopteris thalictroides). Samples 13 and 18 are positive plasmid DNA controls, and samples 14 and 16 are non-transgenic control sporophytes.

### DETAILED DESCRIPTION OF THE INVENTION

### I. Definitions

The phrases “P. vittata actin promoter” or “Pteris vittata actin promoter” as used herein refers to any promoter comprising at least the 250 nucleotides of a promoter that are immediately adjacent to the transcribed potions of a P. vittata actin gene that has at least 70%, 85%, 90%, 95%, 98% or 100% sequence identity to the P. vittata actin gene that is operably associated with the SEQ ID NO:1 actin promoter (i.e. has at least 70%, 85%, 90%, 95%, 98% or 100% sequence identity to SEQ ID NO:2).

The phrase “recombinant DNA construct” as used herein refers to any DNA molecule in which two or more ordinarily distinct DNA sequences have been covalently linked by human intervention. Examples of DNA constructs include, but not limited to plasmids, cosmids, viruses, BACs (bacterial artificial chromosome), YACs (yeast artificial chromosome), T-DNA vectors, transposon-based transformation vectors, vectors comprising homologous target host cell DNA sequences that provide for targeted integration into the target host cell genome, minichromosomes, autonomously replicating sequences, phage, or linear or circular single-stranded or double-stranded DNA sequences, derived from any source, that are capable of genomic integration or autonomous replication. DNA constructs can be assembled by a variety of methods including but not limited to recombinant DNA techniques, DNA synthesis techniques, PCR (Polymerase Chain Reaction) techniques, or any combination of techniques.

The phrase “gene that provides for a counter-selection” as used herein refers to any gene that when expressed permits destruction or sterilization of a cell or organism comprising the gene. Such genes include, but are not limited to, a glyphosate phosphonate ester hydrolase (U.S. Pat. No. 5,254,801), an indoleacetamide hydrolase, cytosine deaminase, and a D-amino acid oxidase (Erikson et al., Nature Biotech. 2004, 22, 455-458).

The phrase “gene that provides for inhibition of senescence” as used herein refers to any gene that provides for any delay in onset of senescence in a transgenic fern relative to a control fern that lacks the gene. Senescence symptoms that can be delayed include, but are not limited to, yellowing and/or abscission. Such genes include, but are not limited to, genes that inhibit ethylene biosynthetic enzyme production (i.e. ACC Synthase siRNA inhibitors and the like) genes that inhibit accumulation of ethylene precursors (i.e. ACC deaminases and the like), and genes that inhibit ethylene perception (i.e. siRNAs directed against ethylene receptors, dominant negative forms of ethylene receptors, and the like).

The phrase “a heterologous promoter”, as used herein in the context of a DNA construct, refers to either: i) a promoter that is derived from a source distinct from the operably linked structural gene or ii) a promoter derived the same source as the operably linked structural gene, where the promoter's sequence is modified from its original form.

The phrase “a heterologous signal peptide”, as used herein, refers to a signal peptide that is obtainable from a source other than the native gene.

The term “homolog” as used herein refers to a gene related to a second gene by identity of either the DNA sequences or the encoded protein sequences. Genes that are homologs can be genes separated by the event of speciation (see “ortholog”). Genes that are homologs may also be genes separated by the event of genetic duplication (see “paralog”). Homologs can be from the same or a different organism and may perform the same biological function in either the same or a different organism.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter and is linked such that the resulting translation product is in frame with the translational open reading frame that encodes the protein desired. Nucleic acid sequences that can be operably linked include but are not limited to: sequences encoding peptides that provide for protein localization functions (i.e. signal peptide sequences that provide for secretion of a mature protein, plastid transit peptide sequence that provide for localization of proteins to plastids), sequences that provide gene expression functions (i.e. gene expression elements such as promoters, 5′ untranslated regions, introns, protein coding regions, 3′ untranslated regions, polyadenylation sites, and/or transcriptional terminators), sequences that provide DNA transfer and/or integration functions (i.e. T-DNA border sequences, site specific recombinase recognition sites, integrase recognition sites), sequences that provide for selective functions (i.e. antibiotic resistance markers, biosynthetic genes), sequences that provide scoreable marker functions (i.e. reporter genes), sequences that facilitate in vitro or in vivo manipulations of the sequences (i.e. polylinker sequences, site specific recombination sequences) and sequences that provide replication functions (i.e. bacterial origins of replication, autonomous replication sequences, centromeric sequences).

The phrase “percent identity” as used herein refers to the number of elements (i.e., amino acids or nucleotides) in a sequence that are identical within a defined length of two optimally aligned DNA, RNA or protein segments. To calculate the “percent identity”, the number of identical elements is divided by the total number of elements in the defined length of the aligned segments and multiplied by 100. When percentage of identity is used in reference to proteins it is understood that certain amino acid residues may not be identical but are nonetheless conservative amino acid substitutions that reflect substitutions of amino acid residues with similar chemical properties (e.g., acidic or basic, hydrophobic, hydrophilic, hydrogen bond donor or acceptor residues). Such substitutions may not change the functional properties of the molecule. Consequently, the percent identity of protein sequences can be increased to account for conservative substitutions.

The term “protonemata” as used herein refers to newly germinated spores that have yet to reach maturity as a gametophyte.

The term “transformation” as used herein refers to a process of introducing an exogenous DNA sequence into a cell in which that exogenous DNA is incorporated into a chromosome. In this context an exogenous includes, but is not limited to, a recombinant DNA construct.

The phrase “stably transformed” as used herein in reference to transgenic ferns refers to either a spore, prothallus, gametophyte, or sporophyte that consist of a cell (in a spore) or consist wholly of cells (in a prothallus, gametophyte, or sporophyte) that comprise a recombinant DNA construct, where the spore, prothallus, gametophyte, or sporophyte is capable of yielding a progeny spore population that is transgenic (comprise the recombinant DNA construct) as an adult sporophyte.

The phrase “sensor gene” as used herein refers to a gene that provides for identification of an external stimulus. Such stimuli include, but are not limited to, heavy metals, volatile organic chemicals, halocarbons, and the like.

The term “vector” as used herein refers to a DNA or RNA molecule capable of replication in a host cell and/or to which another DNA or RNA segment can be operatively linked so as to bring about replication of the attached segment. A plasmid is an exemplary vector. Vectors can also comprise sequences that provide for integration into a host cell genome that include, but are not limited to, T-DNA, transposon, and homologous target host cell DNA sequences that provide for targeted integration into the target host cell genome.

As used herein, the phrase “Pteroid fern” consists of Pteris spp., Ochropteris spp., Neurocallis spp., Platyzoma spp., Eriosorus spp., Jamesonia spp., Pterozonium spp., Pityrogramma spp., Actiniopteris spp., and Onychium spp. Such ferns are described in E. Schuettpelz et al., Molecular Phylogenetics and Evolution 44 (2007) 1172-1185.

As used herein, the phrase “Ceratopteridoid fern” consists of Acrostichum spp. and Ceratopteris spp. Such ferns are described in E. Schuettpelz et al., Molecular Phylogenetics and Evolution 44 (2007) 1172-1185.

### Methods for Obtaining Stably Transformed Ferns

Methods for obtaining stably transformed ferns that result in recovery of spores, prothalli, gametophytes, and sporophytes that consist of a cell (in a spore) or wholly of cells (in a prothallus, gametophyte, or sporophyte) that comprise a recombinant DNA construct and that is capable of yielding a progeny spore population that is wholly transgenic (comprise the recombinant DNA construct) as an adult sporophyte are provided herein. Stably transformed ferns provided herein can thus be distinguished from transiently transformed and/or chimeric fern transformants that comprise transiently transformed spores that can not be propagated to obtain an adult sporophyte capable of yielding a progeny spore population that is wholly transgenic (comprise the recombinant DNA construct). Stably transformed ferns provided herein can thus be distinguished from transiently transformed and/or chimeric fern transformants that comprise transiently transformed spores that can not be propagated to obtain an adult sporophyte capable of yielding a progeny spore population that is wholly transgenic (comprise the recombinant DNA construct). Stably transformed ferns provided herein can also thus be distinguished from transiently transformed and/or chimeric prothalli, gametophyte, or sporophyte transformants that comprise transiently transformed spores that can not be propagated to obtain an adult sporophyte capable of yielding a progeny spore population that is wholly transgenic (comprise the recombinant DNA construct) and/or comprise both transformed cells that contain the recombinant DNA construct and untransformed cells that do not contain the DNA construct. Stably transformed ferns such as those provided herein are more useful that transiently transformed and/or chimeric ferns as the stably transformed ferns are more effectively used in various applications that include, but are not limited to, bioremediation, environmental sensing, and horticulture and are more efficiently propagated for such applications.

In the methods provided herein, the recombinant DNA is introduced into a haploid spore or a haploid protonemata cell. Use of spores is advantageous as spores can be germinated and the entire fern life cycle can be generated in vitro. Transformed protonemata cells can also be cultured to yield prothalli which can in turn be cultured to yield all subsequent stages of the fern life cycle. Methods for in vitro propagation of fern spores to yield subsequent life stages that are applicable to propagation of stably transformed transgenic spores include, but are not limited to, Khan et al. Pak. J. Bot., 40(1): 91-97, (2008) for Asplenium, Zheng et al. (2008) Acta Physiologiae Plantarum Volume 30, Number 2, 249-255 for Pteri, and Knauss Proc. Fla. State Hort. Soc. 89:363-365. 1976 for various ferns, Alma G. Stokey (Botanical Gazette (1940) 759-790; and J. H Miller and P. M Miller (1961) American Journal of Botany vol 8 No 2 (1961) 154-159, each of which is incorporated herein by reference in their entirety. Typically, the surface sterilized spores that have been transformed with a recombinant DNA construct are transferred to media comprising Murashige and Skoog (MS) salts at a ½× or 1× concentration and cultured until reaching the gametophyte stage. Transformed gametophytes are then transferred to a potting mix comprising soil or sand/soil mixture. Transfer to a potting mix can be facilitated in certain instances by exposure of gametophytes to an auxin including, but not limited to indolebutryic acid, to induce rooting. Establishment of the transformed gametophytes in soil can be facilitated by initial exposure to MS salts. Propagation of the transferred transformed gametophytes can also be facilitated by provision of liquid fertilizers. Transgenic spores can then be harvested from the adult sporophytes that have been propagated in soil.

Introduction of recombinant DNA constructs into spores is accomplished by surface sterilization of spores, resuspension in a suitable medium, and exposure to a DNA transfer agent. Surface sterilization of spores is typically accomplished by exposure of spores to a dilute aqueous solution of bleach in the presence of a non-ionic detergent. Useful non-ionic detergents include, but are not limited to, Triton™ X100, Brij™ 58, Tween™ 20, and Nonidet™ P-40. Non-ionic detergent concentrations that can be used are typically in a range of about 0.1% to about 0.01% (volume/volume). Suitable media for resuspension include, but are not limited to, sterile aqueous solutions comprising agents such as various water soluble cellulose derivatives. Suitable water-soluble cellulose derivatives include, but are not limited to, hydroxyethylcellulose, hydroxypropylcellulose, and carboxy methyl cellulose. Useful concentrations of the water soluble cellulose derivative include, but are not limited to, a range from any of about 0.5% or 1.0% to about 2.0% or 2.5% (all volume/volume). In certain embodiments, a concentration of about 1.5% carboxy methyl cellulose is used. In certain embodiments, a low viscosity form of a particular water-soluble cellulose derivatives is used. In still other embodiments, low viscosity carboxy methyl cellulose is used. In certain embodiments, a concentration of about 1.5% carboxy methyl cellulose of a low viscosity form is used.

Useful DNA transfer agents include, but are not limited, to Agrobacterium-mediated transformation, Rhizobium-mediated transformation, Sinorhizobium-mediated transformation, particle-mediated transformation, or “whiskers”-mediated transformation. Methods of using bacteria such as Rhizobium or Sinorhizobium to transform plants are described in Broothaerts, et al., Nature. 2005, 10; 433(7026):629-33 and can be adapted for use in obtaining stably transformed ferns in light of the methods and materials disclosed herein. It is further understood that the recombinant DNA construct can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases have been disclosed (U.S. Pat. No. 7,102,055) and can be adapted for use in stably transformed ferns in light of the methods and materials disclosed herein. Such site specific recombination sites can also be used in the recombinant DNA construct to provide for excision of a gene.

Methods for Agrobacterium-mediated transformation of spores are provided herein. In such methods, the surface-sterilized spores are co-cultivated with an Agrobacterium strain comprising a recombinant DNA construct that is a T-DNA vector. Such T-DNA vectors can be either co-integrate-type T-DNA vectors (U.S. Pat. No. 4,693,976) or binary vectors (Bevan, Nucleic Acids Res. 1984 Nov. 26; 12(22): 8711-8721; Komori et al. Plant Physiology 145:1155-1160 (2007)). Agrobacterium-mediated plant transformation vectors typically comprise sequences that permit replication in both E. coli and Agrobacterium as well as one or more “border” sequences positioned so as to permit integration of the expression cassette comprising gene(s) of interest into the fern chromosome. Such Agrobacterium vectors can be adapted for use in either Agrobacterium tumefaciens or Agrobacterium rhizogenes. Selectable markers encoding genes that confer resistance to antibiotics are also typically included in the vectors to provide for their maintenance in bacterial hosts.

In the methods provided herein, transformation of fern spores with Agrobacterium can in certain embodiments include co-cultivation of the Agrobacterium and spores in a medium that contains an agent that provides for induction of the Agrobacterium vir genes that provide for transfer of the T-DNA to the fern spore genome. Useful vir gene inducing agents include, but are not limited to, acetosyringone, alpha-hydroxy acetosyringone, coniferin, synaptic acid methyl ester, ethyl ferulate, sinapyl alcohol, and coniferal alcohol (see Xu et al. Plant Cell Reports Volume 12, Number 3, 160-164, 1992). An acidic environment of less than pH 6.0 can also promote vir gene induction (Stachel et al., Proc Natl Acad Sci USA. 1986). In certain embodiments, acetosyringone is used as an inducing agent. Concentrations of acetosyringone of about 150 micromolar to about 250 micromolar can be used. A concentration of about 200 micromolar acetosyringone is also effective.

Particle-mediated transformation of both spores and protonemata are also provided herein. Preparation of particles suitable for transfer of DNA into cells by microprojectile bombardment and microprojectile bombardment techniques can be achieved essentially as described in U.S. Pat. Nos. 5,879,918, 5,120,657, and 5,506,125. Particles can be comprised of gold, tungsten or other suitable materials. Spores are prepared for bombardment via surface sterilization and resuspension in suitable media as described above. After two weeks, the spores germinate to form protonemata which are suitable for transformation.

In other embodiments, a scoreable marker gene that provides for identification of a transgenic fern can be used to identify transformants. Scoreable marker genes include, but are not limited to, genes that encode enzymes or fluorescent proteins. Useful enzymes that can be used as scoreable markers for transformation include, but are not limited to, a beta-glucuronidase protein (GUS), a beta-galactosidase protein, a luciferase protein derived from a luc gene, a luciferase protein derived from a lux gene, a sialidase protein, streptomycin phosphotransferase protein, a nopaline synthase protein, an octopine synthase protein, anthocyanin biosynthetic enzymes or positive transcriptional regulators of the same, and a chloramphenicol acetyl transferase protein. Useful fluorescent proteins that can be used as scoreable markers include, but are not limited to, a green fluorescent protein (GFP), a yellow fluorescent protein (YFP), or red fluorescent protein (RFP). Scoreable markers that provide for changes in pigmentation including, but not limited to, anthocyanin biosynthetic enzymes or positive transcriptional regulators of the same, are described in US Patent Application Publication 20050114923, which is incorporated herein by reference in its entirety. Ferns transformed with the scoreable marker are identified by assaying for the presence of the scoreable marker. Transformed ferns that contain the scoreable marker can then be manually selected for further propagation.

In certain embodiments, transgenic ferns can be obtained by linking the gene of interest to a selectable marker gene, introducing the linked transgenes into the spore or protonemata by any one of the methods described above, and recovering the transgenic fern under conditions requiring expression of said selectable marker gene for growth of a spore or other subsequent fer-lifecycle stage. The selectable marker gene can be a gene encoding a neomycin phosphotransferase protein, a plant ATP binding cassette (ABC) transporter gene (US Patent Application Publication 20080250527), a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, and an aminoethylcysteine insensitive octopine synthase protein. The corresponding selective agents used in conjunction with each gene can be: neomycin (for neomycin phosphotransferase protein selection), phosphinotricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

In certain embodiments, methods of fern transformation provided herein can comprise culturing spores, protonemata, or other life cycle stages on a surface that is permeable and transferable. At least one advantage of using a permeable and transferable surface is that the surface facilitates parallel processing and/or treatment of fern spores, protonemata, or other fern life cycle stages (i.e. prothalli, gametophytes, and sporophytes) at various transformation method stages. In particular, use of the permeable and transferable surface can obviate laborious manual transfer steps of individual spores, protonemata, prothalli, gametophytes, and sporophytes that are time consuming and, in some instances, potentially injurious. In general, the permeable and transferable surface is any surface that can be placed on a solid media that will permit transfer of water soluble materials present in that media to fern spores, protonemata, or other life cycle stages that are growing on the opposite face of the surface. The surface should permit transfer of materials in the media that include, but are not limited to, organic and inorganic salts, sugars, antibiotics, selectable agents, and scoreable agents. The composition of the surface is also such that it is not detrimental to growth of the applicable fern life cycle stage (i.e. spore, protonemata, prothallus, gametophyte, or sporophyte). In certain embodiments, the surface can comprise a membrane that includes, but is not limited to, a polyvinylidene fluoride (PDVF) membrane, a nitrocellulose membrane, a cellulose acetate membrane, and a polytetrafluoroethylene (PTFE) membrane. Transfer of the surface to fresh media and/or to a distinct media can be accomplished with forceps, spatulas, or other suitable devices that are either sterile or sterilizable.

### Recombinant DNA Constructs for Fern Transformation

Provided herein are recombinant DNA constructs suitable for transformation of ferns that comprise expression cassettes where various promoter, coding, and polyadenylation sequences are operably linked. In general, expression cassettes typically comprise a promoter that is operably linked to a sequence of interest which is operably linked to a polyadenylation or terminator region. In certain instances, it may also be useful to include one or more intron sequences. In particular, inclusion of an intron can facilitate identification of transformed ferns in instances where a scoreable marker is used in an Agrobacterium-mediated transformation vector. Such introns can distinguish undesirable scorable marker gene expression that may occur in the Agrobacterium host from the desirable expression of the scoreable marker gene that is associated with integration of the gene into the fern cell genome. When an intron sequence is included it is typically placed in the 5′ untranslated leader region of the transgene. However, one or more introns can also be placed at other locations, including a coding region, so long as the reading frame of the desired gene product is not disrupted or so long as the resultant gene product possesses the desired activity or characteristics. In certain instances, it may also be useful to incorporate specific 5′ untranslated sequences in a transgene to enhance transcript stability or to promote efficient translation of the transcript.

A variety of promoters can be used in the practice of this invention. One broad class of useful promoters are referred to as “constitutive” promoters that are active in a wide variety of fern plant cell types. For example, the promoter can be a viral promoter derived from a plant Caulimo virus such as a CaMV35S or FMV35S promoter. Enhanced or duplicate versions of the CaMV35S and FMV35S promoters can be used to express a gene of interest in ferns (U.S. Pat. No. 5,378,619, incorporated herein by reference in its entirety). It is understood that this group of exemplary promoters is non-limiting and that one skilled in the art could employ other promoters that are not explicitly cited here in the practice of this invention.

A particularly useful promoter that is provided herein is a Pteris vittata actin promoter. A Pteris vittata actin promoter refers to any promoter comprising at least the 250 nucleotides of a promoter that are immediately adjacent to the transcribed portions of a P. vittata actin gene that has at least 70%, 85%, 90%, 95%, 98% or 100% sequence identity to the P. vittata actin gene that is operably associated with the SEQ ID NO:1 actin promoter (i.e. the Actin gene provided as SEQ ID NO:2 in the Table provided herein in Example 6). In certain embodiments, a Pteris vittata actin promoter of the invention can comprise a deletion of about 1 to 10, 50, 100, 200, 250, 300, 350, 400, 500, or 600 nucleotides of the 5′ end of the Pteris vittata actin promoter of SEQ ID NO:1. In certain embodiments, a Pteris vittata actin promoter of the invention can comprise one or more internal deletions of about 1 to 10, 20, 50, 100, 200, 300, 400, 500, or 600 nucleotides of SEQ ID NO:1. In certain embodiments, a Pteris vittata actin promoter of the invention can comprise one or more internal insertions of about 1 to 10, 10 to 20, 20 to 50, 50 to 100, 100 to 200, 200 to 300, 300 to 400, or 400 to 500 heterologous nucleotides into SEQ ID NO:1. In certain embodiments, a Pteris vittata actin promoter of the invention can comprise one or more substitutions of about 1 to 10, 10 to 15, 15 to 20, or 20 to 25 nucleotides of SEQ ID NO:1. In these contexts, it is understood that 1 to 10, 10 to 15, 15 to 20, 20 to 25, 10 to 20, 20 to 50, 50 to 100, 100 to 200, 200 to 300, 300 to 400, or 400 to 500 refer to all whole number integers between and including 1, 10, 15, 20, 25, 50, 100, 200, 300, 400, and/or 500. In certain embodiments, a Pteris vittata actin promoter of the invention can comprise any combination of the aforementioned deletions, substitutions, and/or insertions. Thus, in certain embodiments, a Pteris vittata actin promoter of the invention can also comprise one or more of a deletions, substitution, and/or insertion of 1 to 5 or 1 to 10 nucleotides. In certain embodiments, a Pteris vittata actin promoter of the invention can comprise a promoter sequence that is capable of hybridizing under stringent hybridization conditions to the corresponding residues 1 to 862, 250 to 862, 400 to 862, 500 to 862, or 600 to 862 of SEQ ID NO:1. In this context, “stringent hybridization” conditions are defined as hybridization buffer consisting of 5×SSC at a temperature of 65 degree C., where 1×SSC is 0.15 M NaCl, 0.015 M-trisodium citrate, pH 7.

Another class of useful promoters in the context of this invention are promoters that are induced by various environmental stimuli. Promoters that are induced by environmental stimuli, include but are not limited to, promoters, induced by: i) inorganic compounds or elements; ii) organic compounds; iii) an abiotic stress including but not limited to drought, cold, heat, or osmotic stress; iv) a biotic stress including, but not limited to, fungal infection, insect infestation, viral infection, or herbivore depredation; and v) a biological or chemical warfare agent. In this context, inorganic compounds or elements include, but are not limited to, a metalloids, or a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium, and zinc. In this context, organic compounds include, but are not limited to, volatile organic compounds such as benzene or formaldehyde, halogenated organic compounds such as a polychlorinated biphenyl (PCB), a polychlorinated hydrocarbon (PCH), a chlorofluorocarbon (Freon), a chlorofluoro-hydrocarbon, or a dioxin, a furan, a polycyclic aromatic hydrocarbon or an ether. In this context, chemical warfare agents include, but are not limited to, sarin, ricin, mustard gas, or phosgene. In this context, biological warfare agents include, but are not limited to, anthrax bacilli, anthrax spores, hemorrhagic viruses, or a smallpox virus. Certain environmentally inducible promoters useful for induction by inorganic compounds or elements, organic compounds, or biological or chemical warfare agents are described in US Patent Application Publication 20050114923, which is incorporated herein by reference in its entirety. P. vittata is known to hyper-accumulate arsenic in higher amounts compared to any plants (Dhankher et al., 2006). Under the tested condition it was tolerant of and actively growing in soils containing as much as 1500 ppm arsenic, whereas other plants cannot survive 50 ppm arsenic. This fern is thus a model to study arsenic hyper-accumulation, translocation, and detoxification. Naturally it serves as a great resource to study arsenic phytoremediation. By using transformation techniques provided for herein a fern that can sense as well as phytoremediate arsenic can be generated.

Also contemplated herein are use of genes that can provide for removal of an environmental contaminant and/or use of a gene that provides for detoxification of an environmental, contaminant. Such genes include, but are not limited to, genes that remove or detoxify metals such as metallothioneins, phytochelatins, selenocysteine methyltransferase, and glutathione (Martinez M. et al. 2006. Chemosphere 64: 478-485; Krämer U. 2005. Curr. Op. Biotech. 16: 133-141; Gratão L. P. et al. 2005. Braz. J. Plant Physiol. 17: 53-64; Cherian S. & Oliveira M. 2005. Am. Chem. Soc. 39: 9377-9390). Such genes also include, but are not limited to, certain P450 genes that provide for herbicide detoxification (Gratão L. P. et al. 2005. Braz. J. Plant Physiol. 17: 53-64; Cherian S. & Oliveira M. 2005. Am. Chem. Soc. 39: 9377-9390). Such genes also include, but are not limited to, the bacterial mer A and merB genes that provide for detoxification of organomercurials (Gratão L. P. et al. 2005. Braz. J. Plant Physiol. 17: 53-64; Cherian S. & Oliveira M. 2005. Am. Chem. Soc. 39: 9377-9390; Bizily S. P. et al. 2000. Nature Biotech. 18: 213-217). Such genes also include, but are not limited to, the combination of the bacterial arsC and y-ECS that provide for arsenic removal (Cherian S. & Oliveira M. 2005. Am. Chem. Soc. 39: 9377-9390). Such genes also include, but are not limited to, genes such as the xplA gene of Rhodococcus that provide for resistance to explosives such as RDX and genes such as a NADPH-dependent nitroreductase that provide for both degradation of, and tolerance to, TNT (Rylott E. L., et al. Nature Biotech. 24: 216-219; Cherian S. & Oliveira M. 2005. Am. Chem. Soc. 39: 9377-9390).

An intron may also be included in the DNA expression construct. Introns that can be used include the maize hsp70 intron (U.S. Pat. No. 5,424,412; incorporated by reference herein in its entirety), the maize ubiquitin intron, the Adh intron 1 (Callis et al., 1987), the sucrose synthase intron (Vasil et al., 1989), the rice Act1 intron (McElroy et al., 1990), the CAT-1 intron (Cazzonnelli and Velten, 2003), the pKANNIBAL intron (Wesley et al., 2001; Collier et al., 2005), the PIV2 intron (Markin et al., 1997) and the “Super Ubiquitin” intron (U.S. Pat. No. 6,596,925, incorporated herein by reference in its entirety; Collier et al., 2005). The catalase intron in the pCAMBIA1305.1 (GenBank Accession Number AF354045) and pCAMBIA1305.2 (GenBank Accession Number AF354046) vectors can also be used. It is understood that this group of exemplary introns is non-limiting and that one skilled in the art could employ other introns that are not explicitly cited here in the practice of this invention.

Certain embodiments of this invention comprise a sequence encoding a heterologous signal peptide that allows for secretion of the mature protein from the transformed fern cells. Useful heterologous signal peptide encoding sequences include, but are not limited to, the signal peptide derived from a barley cysteine endoproteinase gene (Koehler and Ho, 1990) or the tobacco PR1b signal peptide. The rice glycine rich protein (GRP) signal peptide in the pCAMBIA1305.2 (GenBank Accession Number AF354045) vector can also be used. It is understood that this group of exemplary heterologous signal peptides is non-limiting and that one skilled in the art could employ other heterologous signal peptides that are not explicitly cited here in the practice of this invention.

Most genes that are readily expressed in most dicot or monocot plants will also be expressed in ferns. In certain embodiments, particularly where the gene of interest is obtained from an organism with a codon usage pattern that is distinct from that of monocot or dicot plants or ferns, the nucleotide coding sequence that is operably linked to the promoter and other expression elements can be designed so that it will be expressed more readily in ferns. In general, the non-native nucleotide sequence will comprise one or more codons that are more abundant (i.e. occur more frequently) in monocot plant, dicot plant, or fern genes. In certain embodiments, greater than at least 25%, 50%, 70%, 80%, or 90% of the codons used in the non-native—encoding nucleotide sequence are codons that are more abundant in fern plant genes. Computer programs such as “BackTranslate” (GCG™ Package, Acclerys, Inc. San Diego, Calif.) can be used to convert a peptide sequence to the corresponding nucleotide sequence with the desired codon usage that encodes the protein of interest. Codon usage in model fern Ceratopteris richardii genes are provided in the table below and can be used to modify coding regions of interest as necessary.

TABLE 1
Codon Usage in Ceratopteris richardii [gbpln]: 38 CDS's (12180 codons)

| | | | | | | | |
|---|---|---|---|---|---|---|---|
| UUU | 17.3(211) | UCU | 16.0(195) | UAU | 16.7(204) | UGU | 7.9(96) |
| UUC | 15.7(191) | UCC | 10.3(126) | UAC | 12.1(147) | UGC | 10.8(131) |
| UUA | 9.8(119) | UCA | 15.8(193) | UAA | 0.7(8) | UGA | 1.3(16) |
| UUG | 19.5(238) | UCG | 6.4(78) | UAG | 1.1(14) | UGG | 10.4(127) |
| CUU | 23.6(288) | CCU | 15.1(184) | CAU | 17.3(211) | CGU | 9.8(119) |
| CUC | 14.1(172) | CCC | 7.6(92) | CAC | 8.2(100) | CGC | 5.7(69) |
| CUA | 8.6(105) | CCA | 15.5(189) | CAA | 19.7(240) | CGA | 8.4(102) |
| CUG | 17.0(207) | CCG | 4.4(54) | CAG | 23.6(288) | CGG | 5.9(72) |
| AUU | 22.2(271) | ACU | 14.6(178) | AAU | 23.2(283) | AGU | 14.2(173) |
| AUC | 15.4(187) | ACC | 11.7(142) | AAC | 17.2(210) | AGC | 12.4(151) |
| AUA | 14.1(172) | ACA | 18.4(224) | AAA | 25.5(310) | AGA | 15.1(184) |
| AUG | 28.8(351) | ACG | 6.2(76) | AAG | 36.9(449) | AGG | 13.4(163) |
| GUU | 20.7(252) | GCU | 23.5(286) | GAU | 38.6(470) | GGU | 18.6(227) |
| GUC | 11.7(143) | GCC | 12.9(157) | GAC | 20.2(246) | GGC | 13.0(158) |
| GUA | 12.9(157) | GCA | 26.5(323) | GAA | 33.6(409) | GGA | 19.6(239) |
| GUG | 20.7(252) | GCG | 8.2(100) | GAG | 41.1(500) | GGG | 12.4(151) |

1) fields: [triplet] [frequency: per thousand] ([number])
2) Coding GC 46.19% 1st letter GC 53.88% 2nd letter GC 39.20% 3rd letter GC 45.50%
3) From the World Wide Web (Internet) at kazusa.or.jp/codon/cgi-bin/showcodon.cgi?species = 49495 and Codon usage tabulated from the international DNA sequence databases: status for the year 2000. Nakamura, Y., Gojobori, T. and Ikemura, T. (2000) Nucl. Acids Res. 28, 292).

As noted above, the sequence of interest may also be operably linked to a 3′ non-translated region containing a polyadenylation signal. This polyadenylation signal provides for the addition of a polyadenylate sequence to the 3′ end of the RNA. The Agrobacterium tumor-inducing (Ti) plasmid nopaline synthase (NOS) gene 3′ and the pea ssRUBISCO E9 gene 3′ un-translated regions contain polyadenylate signals and represent non-limiting examples of such 3′ untranslated regions that can be used in the practice of this invention. It is understood that this group of exemplary polyadenylation regions is non-limiting and that one skilled in the art could employ other polyadenylation regions that are not explicitly cited here in the practice of this invention.

The DNA constructs that comprise the plant expression cassettes described above are typically maintained in various vectors. Vectors contain sequences that provide for the replication of the vector and covalently linked sequences in a host cell. For example, bacterial vectors will contain origins of replication that permit replication of the vector in one or more bacterial hosts. Such DNA vectors can also comprise sequences that facilitate transfer and/or integration of one or more gene(s) of interest to the fern cell genome. In this regard, one or more gene(s) of interest can be flanked by T-DNA border sequences in embodiments in an Agrobacterium-mediated transformation vector. Such vectors can also comprise or more gene(s) of interest flanked by cis-acting site-specific recombination sites recognized by site-specific recombinases, including Cre, Flp, Gin, Pin, Sre, pinD, Int-B13, and R in an orientation suitable for either: i) integration into a cognate site-specific recombination site introduced into the fern genome by transformation or ii) excision via the fern genome after integration. In other embodiments, a gene of interest can also be flanked by homologous target host cell DNA sequences that provide for targeted integration into the target host cell genome (Wright et al., Plant J. 44, 693, 2005; D'Halluin, et al., Plant Biotech. J. 6:93, 2008). In certain embodiments, a homologous replacement sequence can be placed in the vector to provide for insertion into a targeted nuclease cleavage site by non-homologous end joining or a combination of non-homologous end joining and homologous recombination (reviewed in Puchta, J. Exp. Bot. 56, 1, 2005). In certain embodiments, integration of such sequences into a fern genome can be facilitated by use of a meganuclease (WO/06097853A1, WO/06097784A1, WO/04067736A2; U.S. 20070117128A1) or a zinc finger nuclease (WO 03/080809, WO 05/014791, WO 07014275, WO 08/021207) as has been done in certain plants.

### Stably Transformed Ferns

Also provided herein are stably transformed ferns. Stably transformed transgenic ferns can comprise either a spore, prothallus, gametophyte, or sporophyte that consist of a cell (in a spore) or consist wholly of cells (in a prothallus, gametophyte, or sporophyte) that comprise a recombinant DNA construct, where the spore, prothallus, gametophyte, or sporophyte is capable of yielding a progeny spore population that is transgenic (comprise the recombinant DNA construct) as an adult sporophyte. A spore, prothallus, gametophyte, or sporophyte that consist of a cell (in a spore) or consist wholly of cells (in a prothallus, gametophyte, or sporophyte) that comprise a recombinant DNA construct can be identified by techniques including, but not limited to, assaying expression of reporter genes in the cell or cells of the organism and/or analyzing genomic DNA extracted by the organism. Genomic DNA analyses that demonstrate insertion of the recombinant DNA construct into the genomic DNA include, but are not limited to, genomic DNA blot (Southern analysis), PCR analyses, and/or methods of recovering genomic clones comprising the insertion site of the recombinant DNA and sequence analysis of the same. Such genomic clones can be recovered by techniques including, but not limited to, inverse PCR cloning techniques. Identification of adult sporophytes capable of yielding a progeny spore population that is transgenic can be accomplished methods including, but not limited to, harvesting a progeny spore population and assaying spores for expression of a reporter gene.

### EXAMPLES

The following examples are included to demonstrate certain embodiments of the invention. However, those of skill in the art should, in light of the present disclosure, appreciate that many changes can be made in the specific embodiments that are disclosed and still obtain a like or similar result without departing from the spirit and scope of the invention. Thus, specific structural and functional details disclosed herein in the Examples are not to be interpreted as limiting.

### Example 1

### Actin Promoter Isolation and Vector Construction

The P. vittata actin gene was isolated from genomic DNA using conserved degenerate primers which were conserved across ferns (Selaginella apoda), marine algae, and angiosperms (forward primer-5′-ATGGCNGAYGGNGARGA-3′ (SEQ ID NO:3) ; and reverse primer-5′-GAAGCAYTTGCGRTGSACRAT-3′ (SEQ ID NO:4)). After confirming the actin gene isolation by sequence analyses, the 5′ upstream region was isolated by two step genome walking using the universal Genomewalker™ kit (Clontech, Mountain View Calif.). A 900 by fragment upstream to actin CDS was used as a promoter to drive the expression of a beta-glucuronidase reporter gene GUSPlus (pCAMBIA1305.2 Genbank Accession No. AF354046) such that the promoter drives expression of a transcript comprising a rice glycine rich protein (GRP) signal peptide, a catalase intron, and a beta-glucuronidase reporter gene isolated isolated from a Staphylococcus sp. The actin promoter and GUSPlus were subcloned into pDONRP4P1R and PCR8/GW/TOPO (Invitrogen Carlsbad, Calif. 92008) respectively using the manufacturer's instructions for primer design and cloning. The promoter and the GUSPlus fragments were cloned into Gateway™ binary vector pGW 501 (Nakagawa et al., 2008) using a Multisite gateway three fragment vector construction kit (Invitrogen Carlsbad, Calif. 92008). After sequence confirmation the binary vector pGW501 containing the promoter GUSPlus construct was transformed into A. tumefaciens vir helper EHA105 (Hood et al., 1993) by the freeze thaw method. The same binary vector was used for biolistic transformation.

### Example 2

### A. tumefaciens Transformation of P. vittata Spores

Spores were cleaned through 60 micron nylon mesh and surface sterilized in 2.5% bleach containing about 0.4% (v/v) of Tween-20 for five minutes and washed in sterile water for five times using a table top centrifuge (13,000 rpm for 1 min for each wash). The spores were suspended in 0.5 ml 1.5% CMC (carboxy methyl cellulose low viscosity). The approximate spore concentration was 5,000 spores ml⁻¹.

A 2 ml seed culture of Agrobacterium comprising the binary Agrobacterium transformation vector was started in Agrobacterium growth medium with required antibiotics. In these experiments, the Agrobacterium growth medium comprised KPO₄ buffer, MN buffer (MgSO₄, NaCl), CaCl₂, FeSO₄, salts (H₃BO₃, ZnSO₄, CuSO₄, MnSO₄, Na₂MoO₄), NH₄NO₃, 0.5% glycerol (final con), MES and glucose, which is a media that was described by Utermark and Karlovsky 2008. In contrast, Utermark and Karlovsky used LB media to culture Agrobacterium. In the evening, 250 μl from the 2 ml culture was inoculated into 25 ml of the same medium with antibiotics. The culture was grown until the OD reached at 0.8. Then 10 ml was centrifuged at 4,000 rpm for fifteen minutes and resuspended in 20 ml Vir gene induction (IM) medium with 200 μM acetosyringone. Vir gene induction & co-cultivation (IM) medium comprised Agrobacterium growth medium, 200 μM Acetosyringone with 2 g L⁻¹ gellan gum. The Agrobacterium vir genes were induced for 24 hours by shaking the suspension at 60 rpm at room temperature (25° C.). Then 0.5 ml of induced culture was mixed with 0.5 ml of sterilized spore suspension in 1.5% CMC. This mixture was incubated for 15 min and the whole suspension was plated as 25 μl (˜300 spores plate⁻¹) aliquots on top of hydrophilic PVDF membrane in co-cultivation agar plates containing IM for 72 hours. Then membranes were transferred to ½ MS+20 g sucrose plates containing 400 mg L⁻¹ timentin. The spores were sub-cultured for every two weeks in the same medium.

Media for Agrobacterium transformation is made as follows. The IM media can be made by mixing 800 μl of 1.25 M potassium phosphate buffer, 20 ml MN buffer, 1 ml of 10 mg ml-1 CaCl₂, 1 ml of 1 mg ml-1 FeSO₄, 5 ml IM-salts, 2 ml of 200 mg ml-1 NH₄NO₃, 10 ml of 50% glycerol, 40 ml of 1 M MES and 1 ml (liquid medium) or 5 ml (solid medium) of 200 mg ml-1 glucose and make up the volume to 1000 ml. For solid medium (co-cultivation) add 15 g agar. Autoclave at 121° C. for 15 min. Cool to 55° C., add 1 ml of 200 mM acetosyringone. Mix vigorously. Solid medium: dispense 20 ml per Petri dish. Store the agar plates and liquid medium at room temperature. MN buffer: Dissolve 30 g MgSO₄ 7 H₂O and 15 g NaCl in 900 ml of distilled water and make up the volume to 1000 ml. Filter-sterilize (0.2 μm filter) and store at room temperature (about 25° C.). The IM-salts can be made by dissolving 100 mg of each H₃BO₃, ZnSO₄ 7H₂O, CuSO₄ 5H₂O, MnSO₄ H2O, and Na₂MoO₄ 2H2O in 900 ml of distilled water and make up the volume to 1000 ml. This is filter-sterilized (0.2 μm) and stored at room temperature.

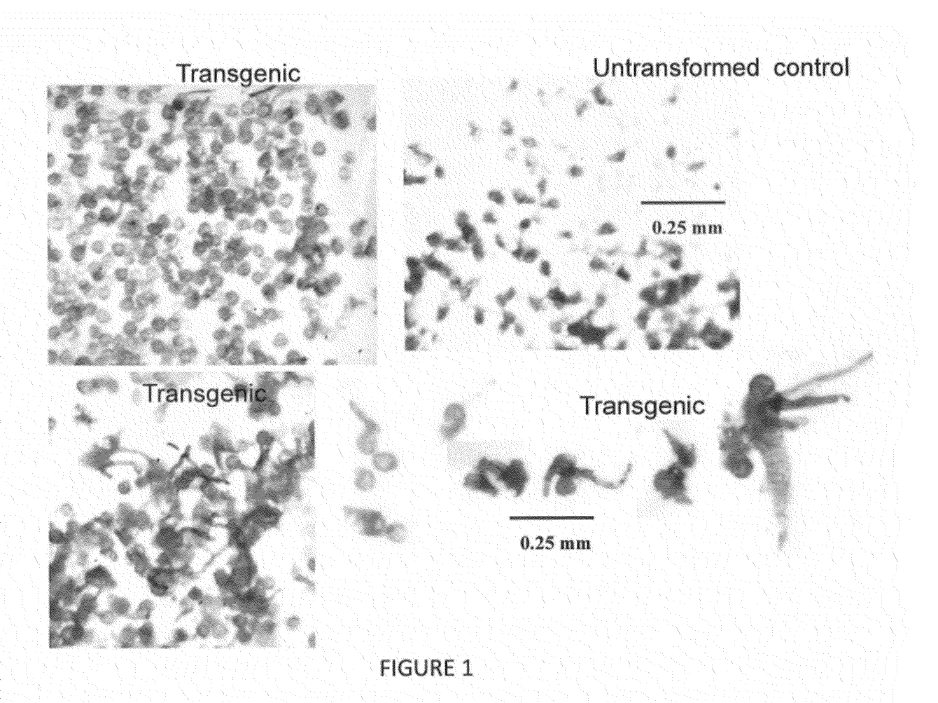

Stable recombinant beta-glucuronidase (GUS) reporter gene expression of Agrobacterium transformed spore derived prothalli was observed at four weeks after transformation (FIG. 1). Beta-glucuronidase (GUS) staining to detect reporter gene expression was done using a standard protocol (Jefferson et al., 1987) after pre-incubating the tissues in ice for 30 min with 95% cold acetone. The tissues were destained using 70% ethanol, rinsed with distilled water, and viewed under the microscope. Because the GUSPlus gene has a Oryza sativa signal peptide to localize the expression in the apoplastic region, in almost all the tissues that were screened the GUS expression was localized only in some portions of spore-derived tissues and in immature prothalli. Compared to untransformed control tissues, the transformed spores grew at least two times slower based on their ability to form mature prothalli. This data thus indicates that stable, non-chimeric transformation in Pteris vittata prothalli through Agrobacterium-mediated transformation of spores was achieved.

### Example 3

### Full and Chimeric Transformation of P. vittata Spore and Prothalli Using Biolistic Bombardment

Spores were cleaned through 60 micron nylon mesh and surface sterilized in 2.5% bleach for one minute, 75% ethanol for one minute, and washed in sterile water three times using a table top centrifuge (13,000 rpm for 1 min for each wash). The spore suspension was plated as 25 μl (˜300 spores plate⁻¹) aliquots on top of hydrophilic PVDF membrane in co-cultivation agar plates comprising ½ Murashige and Skoog (MS) salts+20 g sucrose plates containing 400 mg L-1 timentin. Pteris vittata fronds and Nicotiana tabacum cv. Xanthi leaf controls were also arranged in the same area at the center of medium plates containing only agar. Gold particles (0.6 μm) were sterilized in 100% ethanol and then vector DNA was bound to the gold using CaCl2 and spermidine. The vector used for transformation was pGW501 with a dual (2×) 35S Cauliflower mosaic virus (CaMV) promoter driving the gusA beta-glucuronidase marker gene. Ten day old plates of spores and newly emerging protonemata were bombarded using 7584 kPa (1,100 psi) rupture disks (BioRad™, Hercules, Calif., USA, Cat #1652329). GUS assays were performed on calli three and six days post-transformation. To stain for GUS activity, tissues were put into GUS staining solution (50 mg X-GLUC, 5 ml DMSO, 10 ml 1M KPO4, and 2 drops of Triton-X-100) and vacuum infiltrated for 3 minutes, and then left in a 37° C. oven overnight. The GUS staining solution was then removed using a 3:1 mixture of 100% ethanol:glacial acetic acid.

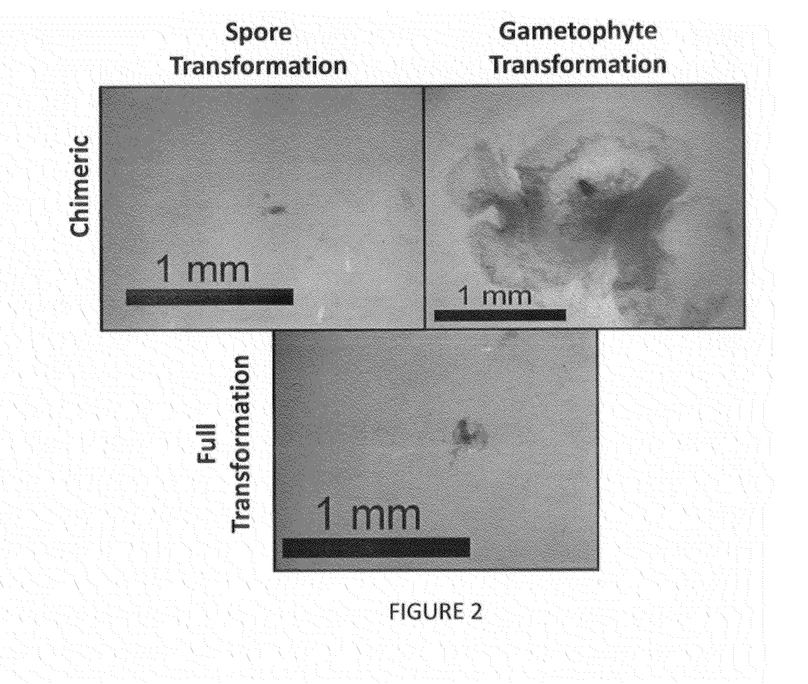

Transformation of P. vittata spores and emerging protonemata resulted in two distinct populations of prothalli (young gametophytes): chimeric and fully transformed (FIG. 2). In comparison, when mature gametophytes were transformed using biolistic bombardment, only small sections of the gametophytes exhibited GUS expression. In fully transformed (i.e. stable transformants) prothalli expression of gusA was seen throughout dividing cells of the protonemata, rhizoids, and gametophyte blade (FIG. 2, bottom panel entitled “Full Transformation). The extent of GUS expression in chimeric prothalli was highly variable as would be expected. Transgenic gametophytes exhibiting stable, non-chimeric expression of a reporter gene were obtained by particle-mediated transformation.

### Example 4

### Propagation and Characterization of Transgenic Spores, Prothalli, and/or Gametophytes Obtainable by Agrobacterium- and/or Particle-Mediated Transformation

The transgenic spores, prothalli, and gametophytes generated in Examples 3, 4 or by essentially similar methods will be germinated and grown to the sporophyte portion of their life cycle in vitro, and then transgene integration will be confirmed by Southern (i.e. genomic DNA blot) hybridization analyses. The gametophytes will be transferred to sucrose free ½ (Murashige and Skoog) MS medium for sporophyte induction. The inheritance of transgenes will be tested in the next generation spores by PCR, reporter gene expression (including but not limited to GUS expression), and Southern (i.e. genomic DNA blot) hybridization analyses.

### Example 5

### Transformation of Ferns with New Vectors and Transformation of Additional Fern Types

The spores will be transformed with a simpler reporter gene, GUSPlus (with an intron but without the apoplastic signal peptide) from pCAMBIA1305.1 (GenBank Accession No. AF354045) to more accurately determine GUS localization in expressing tissues. Fluorescent proteins like tdTomato and GFP will be also tried as reporter genes. As a control for the actin promoter the dual (2×) CaMV35S promoter will be used for reporter gene expression. A. tumefaciens vir helper EHA105 will be used in the majority of the Agrobacterium-mediated transformation experiments. Initially a hygromycin resistance gene will be used as a selectable marker for transformation. If the fluorescent protein expression is highly reproducible then hygromycin selection method will be discontinued.

The Agrobacterium- and/or particle-mediated transformation methods described herein will also be applied to other ferns that include: Polystichum acrostichoides (Christmas fern), Asplenium platyneuron (Ebony spleenwort), Asplenium nidus (Japanese bird's nest fern), Onoclea sensibilis (Sensitive fern), Pteris cretica, Pteris ensiformis, and Adiantum raddianum (maidenhair fern).

### Example 6

### Sequences

| SEQ | | |
|---|---|---|
| ID | PROMOTOER, GENE, OR | |
| NO: | OLIGONUCLEOTIDE | SEQUENCE |
| 1 | P. vitatta Actin Promoter of SEQ ID | AATCATTTTGGATATGTCAAATATTAAATTTT |
| | NO: 1 | ATGACATGACGTATGCGACATAGAGTTAATG |
| | | CAACTGCTCCCAATATATACATTACTAAAAG |
| | | TAGACAGTGTGATAGTGCTGCCCCATCCCTCT |
| | | CGTTATATCCCGTTCTGAACACAGGATTTGTG |
| | | ATGAATATACTTGGATTATCCAAGGCCGATG |
| | | CCAACATTACCTATGCCTGACTGCCAGCCAA |
| | | GTAACACAGCTTATGTGTGGCAGTGTCAGAG |
| | | AGTGGTCGTAGTGAGGGTATTGTATAGAGTA |
| | | GTACTAGATATAGGAAGTATGTATTTTCCTAT |
| | | GGGGCCAAGGTTTCTAAATCTGTGTGTGGTA |
| | | TTAACTATACACTATGGATAACGAAGACAGT |
| | | TAATTTTCAGAAGATATTAAACAACGCGAAA |
| | | ATTCAGTAGTAGCGAATGTCGATTGTTAGTG |
| | | CATGCACCTCAGTAGGGATCAGACCCTACTT |
| | | GTGTGGTTGTGTTGGATGCCATTTGCGAGTTT |
| | | GCAATGTCAGCACTTCAGTTGCTACCACCGA |
| | | TTATTCGATATGTTTGTCAAAGCTGTTGTATA |
| | | TCGATGCGTGTGTAGTCTTTTTCCCGCCCAGG |
| | | ATGATAAAGATGTGGGTTGATCCACGCGCGC |
| | | ACGTGCGTGTGTGTGTGAGATAGGTAGATTG |
| | | AACTGCACGGGGGGCAATGTCCTTGCAGTGA |
| | | TTGATTAGTATGCGTGTGAGGGTTAATTGTG |
| | | GGTGTGTGAGTGTGAATATACTAGACAAAGG |
| | | TGCCACGTATTTCTGTAATACGTGTTGTGAAA |
| | | GATGTTGCCAGAGCGTAGCAGGAGCTGGAAG |
| | | TTGTCACAATCAGAAGAGAGCAGCATCTACG |
| | | TGCGTGCATGTGGGGAG |
| 2 | P. vitatta Actin Gene Associated with the | ATGGATCAGGAATGGTGAAGGTGAGATGCTT |
| | Promotoer of SEQ ID NO: 1 | CACTCTCTCCATGGTTTTTGTCCTTGCAAAGT |
| | | GTCAATGCATATTGATTTGATGGTTCTATTTC |
| | | TACTTTCTTTTGCCCTGGAGGTCAGGCTGGAT |
| | | TTGCTGGTGATGATGCCCCAAGGGCTGTTTTC |
| | | CCAAGTATTGTGGGTCGTCCAAGACACACTG |
| | | ATGTAATGGTGGGAATGGGACAAAAGGATG |
| | | CGTATGTTGGTGATGAGGCACAGTCCAAGCG |
| | | TGGTATTCTTACATTGAAGTATCCTATTGAGC |
| | | ATGGTATTGTCACTAATTGGGATGATATGGA |
| | | GAAGATTTGGCATCATACTTTCTACAATGAG |
| | | CTTCGTGTTGCACCTGAAGAGCATCCCGTTTT |
| | | GCTCACGGAAGCCCCTCTGAATCCCAAGGCA |
| | | AATCGTGAGAAGATGACCCAGATAATGTTTG |
| | | ACACGTTCAATGCTCCAGCAATGTATGTTGCT |
| | | ATCCAGGCTGTGCTCTCCCTATATGCGAGTG |
| | | GAAGAACCACAGGTATGAATGAAATATTGAC |
| | | TGTGATAACTGATTTTGAATTTTGTATTTGCC |
| | | TGCAGAGTAAGTTAACTGAGTTTTTGCCCCTT |
| | | CTGTAGGTATCGTGCTTGATTCCGGTGATGG |
| | | GGTCACGCACACTGTACCCATCTACGAGGGT |
| | | TATGCATTGCCACATGCCATCCTCCGTCTTGA |
| | | TCTTGCTGGCAGGGACCTGACAGATGCCCTT |
| | | ATGAAAATCCTGACTGAGAGAGGGTATTCAT |
| | | TCACAACAACAGCTGAAAGAGAAATTGTTCG |
| | | GGATATCAAAGAGAAGCTAGCATATGTTGCC |
| | | CTGGATTTTGAGCAAGAACTCGAGACTTCAA |
| | | AGAGCAGCTCATCTTTGGAGAAGAATTATGA |
| | | GCTTCCTGATGGGCAGGTCATCACCATCGGT |
| | | GCAGAGCGATTCAGATGTCCAGAGGTTCTCT |
| | | TTCAGCCAGCTCTCATTGGGATGGAAGCTGC |
| | | AGGTATCCATGAGACTACATACAACTCTATC |
| | | ATGAAGTGTGATGTGGATATTCGAAAGGACC |
| | | TGTATGGCAATGTTGTGCTTAGTGGAGGCTC |
| | | CACCATGTTTCCGGGTATTGCTGACCGCATG |
| | | AGCAAAGAGATTACTGCACTTGCTCCTAGCA |
| | | GCATGAAGATAAAGGTTGTGGCACCACCAGA |
| | | GAGGAAGTACAGTGTTTGGATTGGAGGGTCC |
| | | ATTTTGGCATCTTTAAGCACATTCCAACAGGT |
| | | TCTCTACTCAGCTTTTGTAGTCACATTTATTTT |
| | | GTTATGTAGTTTGTGGAGTAACTTTTAGTACT |
| | | TTATGTCATGTTTCTGTTTAAAGGGAAACCTA |
| | | ATTGCTATTTTTCTTTGTGCTTTTTTACAGATG |
| | | TGGATTGCAAAGTCTGAGTATGATGAGTCAG |
| | | GCCCATCCATCGTCCATCGCAAATGCTTC |
| 3 | Forward Primer | ATGGCNGAYGGNGARGA |
| | (Y = T or C; N = A,T,G, or C; R = G or A) | |
| 4 | Reverse Primer | GAAGCAYTTGCGRTGSACRAT |
| | (Y = T or C; N = A,T,G, or C; R = G or A; | |
| | S = G or C) | |

Sequences with homology to the P. vitatta Actin Gene (sequence available on the world wide web at “ncbi.nlm.nih.gov” (National Center for Biotechnology Information).

Sequences producing significant alignments:

| | | Max | Total | Query | | Max |
|---|---|---|---|---|---|---|
| Accession | Description | score | score | coverage | E value | ident |
| GU830959.1 | Pyrus x bretschneideri actin 2 | 991 | 991 | 78% | 0.0 | 80% |
| | gene, complete cds | | | | | |
| AC211306.1 | Populus trichocarpa clone | 987 | 987 | 78% | 0.0 | 79% |
| | POP004-K18, complete | | | | | |
| | sequence | | | | | |
| AM465189.1 | Vitis vinifera contig | 971 | 971 | 78% | 0.0 | 79% |
| | VV78X204398.3, whole | | | | | |
| | genome shotgun sequence | | | | | |
| AC120533.4 | Oryza sativa Japonica Group | 928 | 928 | 78% | 0.0 | 78% |
| | chromosome 11 clone | | | | | |
| | OSJNBa0081F16 map | | | | | |
| | E50055S, complete sequence | | | | | |
| AF285176.1 | Musa ABB Group actin | 913 | 913 | 78% | 0.0 | 78% |
| | (ACT1) gene, complete cds | | | | | |
| EU648503.1 | Psiguria umbrosa clone 09 | 830 | 830 | 71% | 0.0 | 78% |
| | Psig61-65C region genomic | | | | | |
| | sequence | | | | | |
| EU648465.1 | Psiguria bignoniacea clone 08 | 830 | 830 | 71% | 0.0 | 78% |
| | Psig61-65C region genomic | | | | | |
| | sequence | | | | | |
| EU648463.1 | Psiguria bignoniacea clone 06 | 830 | 830 | 71% | 0.0 | 78% |
| | Psig61-65C region genomic | | | | | |
| | sequence | | | | | |
| EU648462.1 | Psiguria bignoniacea clone 05 | 830 | 830 | 71% | 0.0 | 78% |
| | Psig61-65C region genomic | | | | | |
| | sequence | | | | | |
| EU648504.1 | Psiguria umbrosa clone 10 | 827 | 827 | 71% | 0.0 | 78% |
| | Psig61-65C region genomic | | | | | |
| | sequence | | | | | |
| EU648502.1 | Psiguria umbrosa clone 08 | 827 | 827 | 71% | 0.0 | 78% |
| | Psig61-65C region genomic | | | | | |
| | sequence | | | | | |
| EU648497.1 | Psiguria umbrosa clone 03 | 827 | 827 | 71% | 0.0 | 78% |
| | Psig61-65C region genomic | | | | | |
| | sequence | | | | | |
| EU648496.1 | Psiguria umbrosa clone 02 | 827 | 827 | 71% | 0.0 | 78% |
| | Psig61-65C region genomic | | | | | |
| | sequence | | | | | |
| EU648495.1 | Psiguria umbrosa clone 01 | 827 | 827 | 71% | 0.0 | 78% |
| | Psig61-65C region genomic | | | | | |
| | sequence | | | | | |
| EU648477.1 | Psiguria racemosa clone 08 | 827 | 827 | 71% | 0.0 | 78% |
| | Psig61-65C region genomic | | | | | |
| | sequence | | | | | |
| EU648473.1 | Psiguria racemosa clone 04 | 827 | 827 | 71% | 0.0 | 78% |
| | Psig61-65C region genomic | | | | | |
| | sequence | | | | | |
| EU648471.1 | Psiguria racemosa clone 02 | 827 | 827 | 71% | 0.0 | 78% |
| | Psig61-65C region genomic | | | | | |
| | sequence | | | | | |
| EU648464.1 | Psiguria bignoniacea clone 07 | 827 | 827 | 71% | 0.0 | 78% |
| | Psig61-65C region genomic | | | | | |
| | sequence | | | | | |
| EU648460.1 | Psiguria bignoniacea clone 03 | 827 | 827 | 71% | 0.0 | 78% |
| | Psig61-65C region genomic | | | | | |
| | sequence | | | | | |
| EU648512.1 | Ibervillea lindheimeri clone | 825 | 825 | 71% | 0.0 | 78% |
| | 05 Psig61-65C region | | | | | |
| | genomic sequence | | | | | |
| EU648511.1 | Ibervillea lindheimeri clone | 825 | 825 | 71% | 0.0 | 78% |
| | 04 Psig61-65C region | | | | | |
| | genomic sequence | | | | | |
| EU648510.1 | Ibervillea lindheimeri clone | 825 | 825 | 71% | 0.0 | 78% |
| | 03 Psig61-65C region | | | | | |
| | genomic sequence | | | | | |
| EU648490.1 | Psiguria pedata clone 10 | 821 | 821 | 71% | 0.0 | 78% |
| | Psig61-65C region genomic | | | | | |
| | sequence | | | | | |
| EU648458.1 | Psiguria bignoniacea clone 01 | 821 | 821 | 71% | 0.0 | 78% |
| | Psig61-65C region genomic | | | | | |
| | sequence | | | | | |
| EU648508.1 | Ibervillea lindheimeri clone | 820 | 820 | 71% | 0.0 | 78% |
| | 01 Psig61-65C region | | | | | |
| | genomic sequence | | | | | |
| EU648491.1 | Psiguria pedata clone 11 | 818 | 818 | 71% | 0.0 | 78% |
| | Psig61-65C region genomic | | | | | |
| | sequence | | | | | |
| EU648488.1 | Psiguria pedata clone 08 | 818 | 818 | 71% | 0.0 | 78% |
| | Psig61-65C region genomic | | | | | |
| | sequence | | | | | |
| EU648485.1 | Psiguria pedata clone 05 | 818 | 818 | 71% | 0.0 | 78% |
| | Psig61-65C region genomic | | | | | |
| | sequence | | | | | |
| EU648482.1 | Psiguria pedata clone 02 | 818 | 818 | 71% | 0.0 | 78% |
| | Psig61-65C region genomic | | | | | |
| | sequence | | | | | |
| EU648474.1 | Psiguria racemosa clone 05 | 818 | 818 | 71% | 0.0 | 78% |
| | Psig61-65C region genomic | | | | | |
| | sequence | | | | | |
| EU648484.1 | Psiguria pedata clone 04 | 812 | 812 | 71% | 0.0 | 78% |
| | Psig61-65C region genomic | | | | | |
| | sequence | | | | | |
| EU648483.1 | Psiguria pedata clone 03 | 812 | 812 | 71% | 0.0 | 78% |
| | Psig61-65C region genomic | | | | | |
| | sequence | | | | | |
| EU648461.1 | Psiguria bignoniacea clone 04 | 789 | 789 | 68% | 0.0 | 78% |
| | Psig61-65C region genomic | | | | | |
| | sequence | | | | | |
| AB456684.1 | Tulipa gesneriana TgActin1 | 643 | 1026 | 70% | 1e−180 | 83% |
| | mRNA for actin, complete cds | | | | | |
| HM044307.1 | Jatropha curcas actin mRNA, | 636 | 1075 | 77% | 1e−178 | 82% |
| | complete cds | | | | | |
| AF281323.1 | Magnolia denudata actin | 627 | 1014 | 72% | 8e−176 | 82% |
| | mRNA, complete cds | | | | | |
| EF585293.1 | Prunus salicins actin mRNA, | 623 | 943 | 66% | 9e−175 | 82% |
| | partial cds | | | | | |
| FJ560908.1 | Prunus avium actin (CACT1) | 618 | 1067 | 75% | 4e−173 | 84% |
| | mRNA, partial cds | | | | | |
| GU272027.1 | Persea americana actin | 616 | 1091 | 76% | 1e−172 | 83% |
| | mRNA, complete cds | | | | | |
| AF246288.1 | Musa acuminata actin (Mh- | 616 | 1046 | 75% | 1e−172 | 82% |
| | Act1) mRNA, complete cds | | | | | |
| XM_002282480.1 | PREDICTED: Vitis vinifera | 614 | 1067 | 76% | 5e−172 | 82% |
| | actin 1 (ACT1), mRNA | | | | | |
| XM_002331844.1 | Populus trichocarpa actin 9 | 614 | 1103 | 76% | 5e−172 | 83% |
| | (ACT9), mRNA | | | | | |
| FJ410442.1 | Betula luminifera actin | 614 | 1093 | 76% | 5e−172 | 83% |
| | mRNA, complete cds | | | | | |
| EF145186.1 | Populus trichocarpa clone | 614 | 1103 | 76% | 5e−172 | 83% |
| | WS01121_G15 unknown | | | | | |
| | mRNA | | | | | |
| EF144294.1 | Populus trichocarpa clone | 614 | 1103 | 76% | 5e−172 | 83% |
| | PX0015_H06 unknown mRNA | | | | | |
| EF418792.1 | Populus trichocarpa actin | 614 | 1103 | 76% | 5e−172 | 83% |
| | mRNA, complete cds | | | | | |
| AY305730.1 | Gossypium hirsutum actin | 610 | 1060 | 75% | 6e−171 | 82% |
| | (ACT8) mRNA, complete cds | | | | | |
| AY305725.1 | Gossypium hirsutum actin | 610 | 1076 | 76% | 6e−171 | 82% |
| | (ACT3) mRNA, complete cds | | | | | |
| XM_002466911.1 | Sorghum bicolor hypothetical | 609 | 1060 | 77% | 2e−170 | 81% |
| | protein, mRNA | | | | | |
| XM_002298674.1 | Populus trichocarpa actin 1 | 609 | 1091 | 76% | 2e−170 | 84% |
| | (ACT1), mRNA | | | | | |
| EU588981.1 | Betula platyphylla actin | 609 | 1085 | 76% | 2e−170 | 83% |
| | mRNA, complete cds | | | | | |
| FJ560484.1 | Gossypium hirsutum actin 2 | 607 | 1057 | 76% | 7e−170 | 81% |
| | mRNA, complete cds | | | | | |
| FJ560483.1 | Gossypium hirsutum actin 1 | 607 | 1087 | 76% | 7e−170 | 83% |
| | mRNA, complete cds | | | | | |
| AY305737.1 | Gossypium hirsutum actin | 607 | 1066 | 76% | 7e−170 | 81% |
| | (ACT9) mRNA, complete cds | | | | | |
| AY305732.1 | Gossypium hirsutum actin | 607 | 1093 | 76% | 7e−170 | 83% |
| | (ACT11) mRNA, complete cds | | | | | |
| AY305729.1 | Gossypium hirsutum actin | 607 | 1066 | 76% | 7e−170 | 81% |
| | (ACT7) mRNA, complete cds | | | | | |
| AB239789.1 | Rosa hybrid cultivar ACT | 605 | 828 | 59% | 3e−169 | 82% |
| | mRNA for actin, partial cds | | | | | |
| AF112538.1 | Malva pusilla actin (Act1) | 601 | 1033 | 76% | 3e−168 | 81% |
| | mRNA, complete cds | | | | | |
| AY305734.1 | Gossypium hirsutum actin | 601 | 1071 | 76% | 3e−168 | 82% |
| | (ACT13) mRNA, complete cds | | | | | |
| AY305726.1 | Gossypium hirsutum actin | 601 | 1082 | 76% | 3e−168 | 83% |
| | (ACT4) mRNA, complete cds | | | | | |
| AY305724.1 | Gossypium hirsutum actin | 601 | 1069 | 76% | 3e−168 | 83% |
| | (ACT2) mRNA, complete cds | | | | | |
| HP000248.1 | TSA: Arachis duranensis | 600 | 1042 | 76% | 1e−167 | 83% |
| | DurSNP_c248.Ardu mRNA | | | | | |
| | sequence | | | | | |
| GU830958.1 | Pyrus x bretschneideri actin 2 | 600 | 1078 | 76% | 1e−167 | 83% |
| | mRNA, complete cds | | | | | |
| GQ404511.1 | Glycyrrhiza uralensis actin 2 | 600 | 1033 | 76% | 1e−167 | 81% |
| | mRNA, complete cds | | | | | |
| XM_002466915.1 | Sorghum bicolor hypothetical | 600 | 1042 | 77% | 1e−167 | 81% |
| | protein, mRNA | | | | | |
| EU190972.1 | Glycyrrhiza uralensis actin | 600 | 1046 | 76% | 1e−167 | 84% |
| | mRNA, complete cds | | | | | |
| AB190176.1 | Pyrus communis PcACT | 600 | 1037 | 72% | 1e−167 | 84% |
| | mRNA for actin, partial cds | | | | | |
| EZ723877.1 | TSA: Arachis hypogaea | 598 | 1040 | 76% | 4e−167 | 83% |
| | CL1Contig2893.Arhy mRNA | | | | | |
| | sequence | | | | | |
| GU270586.1 | Hevea brasiliensis actin | 598 | 949 | 66% | 4e−167 | 82% |
| | mRNA, partial cds | | | | | |
| AM475102.2 | Vitis vinifera contig | 598 | 989 | 72% | 4e−167 | 81% |
| | VV78X148227.11, whole | | | | | |
| | genome shotgun sequence | | | | | |
| AC087192.14 | Oryza sativa chromosome 10 | 594 | 963 | 72% | 5e−166 | 81% |
| | BAC OSJNBa0005K07 | | | | | |
| | genomic sequence, complete | | | | | |
| | sequence | | | | | |
| EU648253.1 | Psiguria bignoniacea clone 03 | 592 | 592 | 43% | 2e−165 | 81% |
| | Psig129-57C region genomic | | | | | |
| | sequence | | | | | |
| EU648252.1 | Psiguria bignoniacea clone 02 | 592 | 592 | 43% | 2e−165 | 81% |
| | Psig129-57C region genomic | | | | | |
| | sequence | | | | | |
| EU648251.1 | Psiguria bignoniacea clone 01 | 592 | 592 | 43% | 2e−165 | 81% |
| | Psig129-57C region genomic | | | | | |
| | sequence | | | | | |
| AY305727.1 | Gossypium hirsutum actin | 592 | 1051 | 76% | 2e−165 | 83% |
| | (ACT5) mRNA, complete cds | | | | | |
| AJ234400.1 | Hordeum vulgare partial | 592 | 930 | 66% | 2e−165 | 83% |
| | mRNA; clone cMWG0645 | | | | | |
| AF091809.1 | Anemia phyllitidis actin 2 | 592 | 1057 | 76% | 2e−165 | 84% |
| | mRNA, complete cds | | | | | |
| XM_002524970.1 | Ricinus communis actin, | 590 | 1040 | 76% | 6e−165 | 83% |
| | putative, mRNA | | | | | |
| XM_002522148.1 | Ricinus communis actin, | 590 | 1058 | 76% | 6e−165 | 82% |
| | putative, mRNA | | | | | |
| AF246715.1 | Phalaenopsis sp. ‘True Lady’ | 590 | 1033 | 76% | 6e−165 | 82% |
| | actin-like protein (ACT2) | | | | | |
| | mRNA, complete cds | | | | | |
| AY103905.1 | Zea mays PCO141879 mRNA | 590 | 1048 | 77% | 6e−165 | 81% |
| | sequence | | | | | |
| AB473616.1 | Diospyros kaki DkAct1 mRNA | 589 | 999 | 68% | 2e−164 | 83% |
| | for actin-1, partial cds | | | | | |
| AM442507.2 | Vitis vinifera contig | 589 | 952 | 72% | 2e−164 | 80% |
| | VV78X151557.7, whole | | | | | |
| | genome shotgun sequence | | | | | |
| AY305735.1 | Gossypium hirsutum actin | 589 | 1053 | 76% | 2e−164 | 82% |
| | (ACT12) mRNA, complete cds | | | | | |
| FJ869869.1 | Picea abies actin 2 mRNA, | 587 | 1062 | 77% | 7e−164 | 82% |
| | complete cds | | | | | |
| XM_002283554.1 | PREDICTED: Vitis vinifera | 587 | 1055 | 76% | 7e−164 | 84% |
| | hypothetical protein | | | | | |
| | LOC100244452 | | | | | |
| | (LOC100244452), mRNA | | | | | |
| BT071638.1 | Picea sitchensis clone | 587 | 1067 | 77% | 7e−164 | 82% |
| | WS0293_I06 unknown mRNA | | | | | |
| BT071276.1 | Picea sitchensis clone | 587 | 1067 | 77% | 7e−164 | 82% |
| | WS02817_N24 unknown | | | | | |
| | mRNA | | | | | |
| EF677522.1 | Picea sitchensis clone | 587 | 1067 | 77% | 7e−164 | 82% |
| | WS02770_B05 unknown | | | | | |
| | mRNA | | | | | |
| EU648284.1 | Psiguria umbrosa clone 11 | 587 | 587 | 43% | 7e−164 | 81% |
| | Psig129-57C region genomic | | | | | |
| | sequence | | | | | |
| EU648255.1 | Psiguria racemosa clone 02 | 587 | 587 | 43% | 7e−164 | 81% |
| | Psig129-57C region genomic | | | | | |
| | sequence | | | | | |
| NM_001071569.1 | Oryza sativa Japonica Group | 587 | 1019 | 77% | 7e−164 | 81% |
| | Os10g0510000 | | | | | |
| | (Os10g0510000) mRNA, | | | | | |
| | complete cds | | | | | |
| AY360221.1 | Ricinus communis actin | 587 | 1055 | 76% | 7e−164 | 82% |
| | (ACT) mRNA, complete cds | | | | | |
| BT016793.1 | Zea mays done Contig626 | 587 | 1038 | 77% | 7e−164 | 81% |
| | mRNA sequence | | | | | |
| AK101613.1 | Oryza sativa Japonica Group | 587 | 1019 | 77% | 7e−164 | 81% |
| | cDNA clone: J033052E12, full | | | | | |
| | insert sequence | | | | | |
| EU648273.1 | Psiguria pedata clone 09 | 583 | 583 | 43% | 8e−163 | 80% |
| | Psig129-57C region genomic | | | | | |
| | sequence | | | | | |
| EU648269.1 | Psiguria pedata clone 05 | 583 | 583 | 43% | 8e−163 | 80% |
| | Psig129-57C region genomic | | | | | |
| | sequence | | | | | |
| EU648266.1 | Psiguria pedata clone 02 | 583 | 583 | 43% | 8e−163 | 80% |
| | Psig129-57C region genomic | | | | | |
| | sequence | | | | | |
| EU648261.1 | Psiguria racemosa clone 08 | 583 | 583 | 43% | 8e−163 | 80% |
| | Psig129-57C region genomic | | | | | |
| | sequence | | | | | |
| EU648254.1 | Psiguria racemosa clone 01 | 583 | 583 | 43% | 8e−163 | 80% |
| | Psig129-57C region genomic | | | | | |
| | sequence | | | | | |

### Example 7

### Gene Gun and A. tumefaciens Mediated Transformation of Pteris vitta (Chinese Brake Fern) and Ceratopteris thalictroides (C-Fern) Spores

Spores and immature prothalli of Pteris vittata and Ceratopteris thalictroides (C-fern) were used for transformation by biolistics and Agrobacterium tumefaciens respectively. GUS staining analyses of four week old A. tumefaciens transformed tissues derived from spores and sporophytes revealed that stable integration of transgene in immature prothalli and in mature sporophytes (i.e. the last stage of the fern life cycle). PCR analyses of the sporophytes further confirmed the integration of introduced gene in the sporophytes.

Actin promoter isolation and vector construction: The P. vittata actin gene was isolated from genomic DNA using conserved degenerate primers which was conserved across ferns (Selaginella apoda), marine algae, and angiosperms (forward primer-5′-ATGGCNGAYGGNGARGA-3′ (SEQ ID NO:3) and reverse primer-5′-GAAGCAYTTGCGRTGSACRAT-3′ (SEQ ID NO:4). After confirming actin gene isolation by sequence analyses the 5′ upstream region was isolated by two step genome walking using universal genomewalker kit (Clontech, Mountain view Calif.). A 900 by fragment upstream to actin CDS was used as a promoter to drive the expression of a reporter gene GUSPlus (pCAMBIA1305.2 Genbank acc no. AF354046) which contained a castor bean intron and a rice apoplastic signal peptide. The actin promoter and GUSPlus were subcloned into pDONRP4P1R and PCR8/GW/TOPO (Invitrogen, Carlsbad, Calif. 92008) respectively using the manufacturer's instructions for primer design and cloning. The promoter and the GUSPlus fragments were cloned into Gateway binary vector pGW 501 (5)(4) using Multisite gateway three fragment vector construction kit (Invitrogen, Carlsbad, Calif. 92008). After sequence confirmation, the binary vector pGW501 containing the promoter GUSPlus construct was transformed into A. tumefaciens vir helper EHA105 by freeze thaw method. For C-fern transformation, the same P. vittata actin promoter was used to drive GUSPlus gene (pCAMBIA 1305.1 Genbank acc no. AF354045), which does not contain a rice apoplastic signal peptide. For gene gun the binary vector DNA was used as such.

A. tumefaciens Transformation of Spores:

Spores were cleaned through 60 micron nylon mesh and surface sterilized in 2.5% bleach (v/v) containing few drops of Tween-20 for five minutes and washed in sterile water for five times using a table top centrifuge (13,000 rpm for 1 min for each wash). The spores were suspended in 0.5 ml 1.5 CMC (carboxy methyl cellulose low viscosity). The approximate spore concentration was 5,000 spores/ml.

A 2 ml seed culture was started in Agrobacterium growth medium with required antibiotics. In the evening, 250 μl from the 2 ml culture was inoculated to 25 ml of the same medium with antibiotics. The culture was grown until the OD reached at 0.8. About 10 ml was centrifuged at 4,000 rpm for fifteen minutes and resuspended in 20 ml induction (IM) medium with 200 μM acetosyringone. The Agrobacterium vir genes were induced for 24 h by shaking the suspension at 60 rpm at room temperature (25° C.). About 0.5 ml of induced culture was mixed with 0.5 ml of sterilized spore suspension in 1.5 CMC. This mixture was incubated for 15 min and the whole suspension was plated as 25 μl (˜300 spores/plate) aliquots on top of hydrophilic PVDF membrane in co-cultivation agar plates containing IM for 72 h. The membranes were transferred to ½ MS media+20 g sucrose plates containing 400 mg/l Timetin. The spores were sub-cultured for every two weeks in the same medium. The gametophytes which were formed after five weeks (C-fern) or two months (P. vittata) were transferred to ½ MS media (without sucrose) plates (Sheffield, E., et al., 2001. American Fern Journal. 91:179-186) containing 200 mg L-1 timentin for sporophyte development.

Agrobacterium growth medium: KPO₄ buffer, MN buffer (MgSO4, NaCl), CaCl₂, FeSO₄, salts (H₃BO₃, ZnSO₄, CuSO₄, MnSO₄, Na₂MoO₄), NH₄NO₃, 0.5% glycerol (final concentration), MES and glucose as described in (Utermark. J and Karlovsky. P. (2008) Nature protocols. 10.1038/nprot.2008.83).

Vir induction & co-cultivation (IM) medium: Above medium+200 μM Acetosyringone with 2 g/L gellan gum.

Transgene integration analyses: 1. GUS staining: GUS staining was done using standard protocol (Jefferson, R. A., et al. (1987) EMBO J. 6, 3901-3907; Sigma-Aldrich beta-Glucuronidase reporter gene staining kit (Sigma Aldrich, St. Louis, Mo. protocol catalog number GUSS) after pre-incubating the tissues in ice for 30 5 min with 905% methanol.cold acetone. The tissues were destained using 70% methanol or 70% ethanol, rinsed with 50% glycerol and viewed under the microscope.

Genomic PCR:

Genomic DNA from sporophytes was isolated using the CTAB method (Dong, R., et al. (2005). J Ind Microbiol Biotechnol 32: 527-523). About 2-3 fronds from transgenic and non-transgenic control sporophytic tissue were collected and frozen in liquid nitrogen. The tissues were homogenized in 500 μL extraction buffer containing 2% CTAB, 100 mM Tris-HCl pH 8.0, 1.4M NaCl, 20 mM EDTA, 0.2% 2-mercaptoetanol and incubated at 65° C. for 15 min. An equal volume of chloroform isoamyl alcohol (24:1) was then added. The mixture was centrifuged immediately at 10,000×g for 10 min and 500 μL of the supernatant was transferred to a fresh tube, and 500 μL isopropyl alcohol was added and incubated at −20° C. The DNA was pelleted by centrifuging the mixture at 10,000×g, for 10 min. The DNA pellet was washed once in 70% ethanol and resuspended in 100 μL sterile distilled water. 2 μL samples were used for PCR and the rest were stored at −20° C. The PCR was used to confirm the presence of introduced GUSPlus gene in fern sporophytes. The primers FP-5′-CAATTGTCTATGTCAATGGTGAGCTGGTCG-3′ (SEQ ID NO:5) and RP 5′-TGAACATCACTGGATCAATGTCGTGAAAGC-3′ (SEQ ID NO:6) were designed to amplify a 1.2 kb region of GUSPlus gene. DNA purified from plasmid served as a positive control. PCR was carried out in 25 μL volumes using standard protocol. The PCR conditions are as follows: 2 min denaturation at 94° C., followed by 40 cycles of 30 s 94° C., 30 s 55° C., 1 min and 20 s 72° C. and the final extension for 10 min 72° C. The amplified fragments were analyzed using agarose gel electrophoresis.

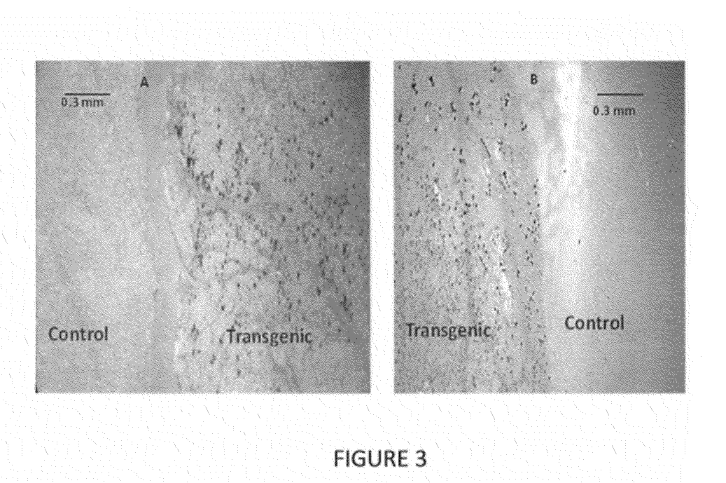

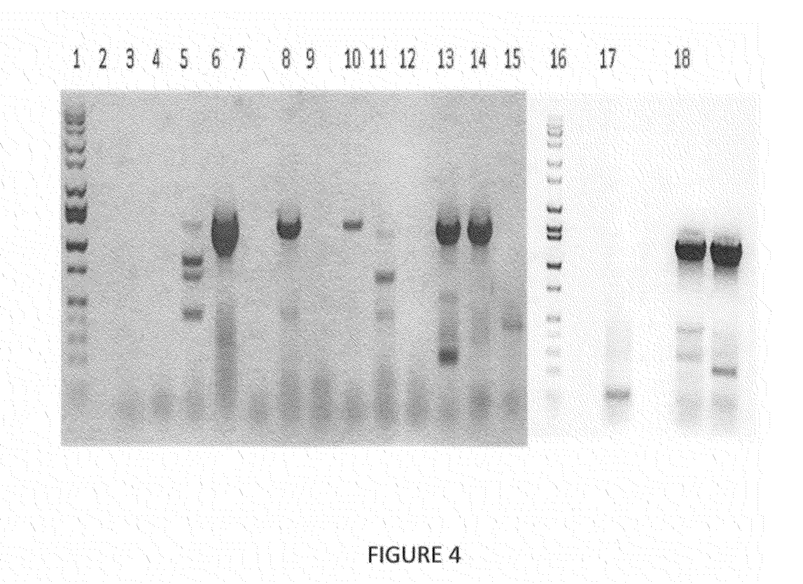

GUS expression was assayed about four weeks after transformation (FIG. 2). GUS expression was also done in mature sporophytes of both P. vittata and C-fern (FIG. 3). Since the GUSPlus gene have rice signal peptide to localize the expression in the apoplastic region, almost all the tissues that were screened GUS expression was localized only in some areas of spore derived tissues and in immature prothalli. Compared to untransformed control tissues the transformed spores were growing at least two times slower based on their ability to form mature prothalli. Both C-fern and P. vittata sporophytes (FIG. 3) showed similar pattern of GUS expression, though C-ferns were transformed with GUS Plus without apoplastic signal peptide. These results suggest that this type of expression could be influenced by P. vittata actin promoter. PCR analyses of transgenic sporophytes also confirmed the presence of GUSPlus transgene in fern genome (FIG. 4). The amplified PCR product was of the expected size (1.2 kb) and was not present in non-transgenic control tissues. In P. vittata six out of ten sporophytes showed the presence of transgene by PCR analysis. In C-fern, the transgene was present in the analyzed sporophyte. These experiments thus demonstrate stable expression of introduced gene through Agrobacterium mediated transformation up to the sporophyte stage (i.e. i.e. final stage of fern life cycle).

The disclosed embodiments are merely representative of the invention, which may be embodied in various forms.

### NON-PATENT PUBLICATIONS

1. Dhankher, O. P., Rosen, B. P., McKinney, E. C., and Meagher, R. B. (2006). Hyperaccumulation of arsenic in the shoots of Arabidopsis silenced for arsenate reductase (ACR2). Proc. Natl. Acad. Sci. USA 103: 5413-5418.
2. Marrs, R. H. & A. S. Watt (2006). Biological Flora of the British Isles 245: Pteridium aquilinum (L.) Kuhn. Journal of Ecology, 94: 1272-1321.
3. Lai H Y, Lim Y Y and Tan S P. (2009). Antimicrobial tyrosinase inhibiting activities of leaf extracts from medicinal ferns. Biosci. Biotechnol. Biochem., 73: 1362-1366.
4. Indriolo, E., Na, G N., Ellis, D., Salt, D. E., and Banks, J. A. (2010). A vacuolar arsenite transporter necessary for arsenic tolerance in the arsenic hyperaccumulating fern Pteris vittata is missing in flowering plants. Plant Cell 10.1105/tpc.109.069773.
5. Ma L Q, Komar K M M, Tu C, Zhang W, Cai Y, Kennelley E D. (2001). A fern that hyperaccumulates arsenic. Nature 409: 579.
6. Nakagawa T., Nakamura S., et al., (2008). Development of R4 gateway binary vectors (R4pGWB) enabling high-throughput promoter swapping for plant research. Biosci. Biotechnol. Biochem., 72: 624-629.
7. Hood, E. E., Gelvin, S. B., Melchers, L. S., and Hoekema, A. (1993). New Agrobacterium helper plasmids for gene transfer to plants. Transgenic Res. 2: 208-218.
8. Utermark. J and Karlovsky. P. (2008). Genetic transformation of filamentous fungi by Agrobacterium tumefaciens. Nature protocols. 10.1038/nprot.2008.83. http://www.natureprotocols.com/2008/03/20/genetic_transformation_of_fila.php
9. Jefferson, R. A., Kavanagh, T. A., and Bevan, M. W. (1987). GUS fusions: β-glucuronidase as a sensitive and versatile gene fusion marker in higher plants. EMBO J. 6, 3901-3907.
10. Mohamed et al. (2006) Transient and stable transformation of the fern Ceratopteris richardii. Plant Biology, Vol. 2006, publication date:AUGUST 2006 PP 312; (Published Abstracts for the Joint Meeting of the American-Society-of-Plant-Biologists/Canadian-Society-of-Plant-Physiology (Plant Biology 2006)).
11. Nugent et al. (2006) Transient and Stable Transformation of the Fern Ceratopteris richardii. Poster Abs #P46051 at the Joint Meeting of the American-Society-of-Plant-Biologists/Canadian-Society-of-Plant-Physiology (Plant Biology 2006) held on Aug. 5-9, 2006. Published online at the http address “abstracts.aspb.org/pb2006/public/P46/P46051.html”

What is claimed is:

1. A transgenic Pteridophyte, wherein said transgenic Pteridophyte is a gametophyte or a sporophyte that is stably transformed with a recombinant DNA construct.

2. The transgenic Pteridophyte of claim 1, wherein said Pteridophyte is Pteris vittata or Ceratopteris thalictroides.

3. The transgenic Pteridophyte of claim 1, wherein said recombinant DNA construct comprises one or more Agrobacterium T-DNA border sequences.

4. The transgenic Pteridophyte of claim 1, wherein said recombinant DNA construct comprises a Pteris vittata actin promoter that is operably linked to a sequence that encodes an RNA, a protein, or both.

5. The transgenic Pteridophyte of claim 1, wherein said recombinant DNA construct comprises a gene that confers resistance to an antibiotic or a herbicide.

6. The transgenic Pteridophyte of claim 5, wherein said herbicide is selected from the group consisting of bromoxynil, dicamba, glufosinate, glyphosate, and sulfonylurea herbicides and wherein said antibiotic is selected from the group consisting of bleomycin, gentamycin, hygromycin, and kanamycin antibiotics.

7. The transgenic Pteridophyte of claim 1, wherein said recombinant DNA construct comprises a sensor gene, a gene that provides for removal of an environmental contaminant, a gene that provides for detoxification of an environmental contaminant, a gene that provides for a counter-selection, a gene that provides for inhibition of senescence, or a combination of said genes.

8. A method of obtaining a transgenic Pteridophyte sporophyte that is stably transformed with a recombinant DNA construct comprising the steps of:
a) introducing a recombinant DNA construct into a spore or a protonemata of a Pteridophyte;
b) isolating a gametophyte obtained from said spore or protonemata that comprises said recombinant DNA construct; and,
c) isolating a sporophyte obtained from said gametophyte, wherein said sporophyte is stably transformed with said recombinant DNA construct.

9. The method of claim 8, wherein said recombinant DNA construct is introduced into said spore by Agrobacterium-mediated transformation or by particle bombardment.

10. The method of claim 8, wherein said recombinant DNA construct is introduced into said protonemata by particle bombardment.

11. The method of claim 8, wherein said recombinant DNA construct comprises a sequence encoding a selectable marker gene that confers resistance to an agent, a sequence encoding a scoreable marker gene, or both of said sequences.

12. The method of claim 11, wherein said agent is an antibiotic or a herbicide.

13. The method of claim 11, wherein said recombinant DNA construct comprises a sequence encoding a selectable marker gene and wherein: i) said isolation in step (b), step (c), or steps (b) and (c) comprises exposure of said gametophyte and/or said sporophyte to an agent that is inhibitory to a gametophyte or a sporophyte that lacks said selectable marker gene or ii) said spore or protonemata is exposed to said agent after introduction of said recombinant DNA construct.

14. The method of claim 8, wherein said spore, said protonemata, or both said spore and said protonemata are cultured on a surface that is permeable and transferable during, following, or both during and following DNA introduction.

15. The method of claim 8, wherein said Pteridophyte is Pteris vittata or Ceratopteris thalictroides.

16. The method of claim 8, wherein said recombinant DNA comprises a Pteris vittata actin promoter that is operably linked to a sequence that encodes an RNA or that encodes an RNA that encodes a protein.

17. A recombinant DNA construct that comprises a Pteris vittata actin promoter that is operably linked to a DNA sequence that encodes a heterologous RNA, or that encodes a heterologous RNA that encodes a heterologous protein.

18. The recombinant DNA construct of claim 17, wherein said promoter comprises residues 1 to 862, 250 to 862, 400 to 862, 500 to 862, or 600 to 862 of SEQ ID NO:1.

19. The recombinant DNA construct of claim 18, wherein said promoter has at least 85%, 90%, 95%, 98%, or 99% nucleotide sequence identity to across the entire length of the corresponding residues 1 to 862, 250 to 862, 400 to 862, 500 to 862, or 600 to 862 of SEQ ID NO:1.

20. The recombinant DNA construct of claim 17, wherein said DNA sequence encodes a heterologous RNA, a heterologous RNA that provides for inhibition of senescence, a heterologous RNA that provides for a counter-selection, or a heterologous protein selected from the group consisting of selectable marker protein, a scoreable marker protein, a protein that provides for a counter-selection, a protein that provides for removal of an environmental contaminant, a protein that provides for detoxification of an environmental contaminant, and a protein that provides for inhibition of senescence.
